(19) United States
(12) Patent Application Publication
(10) Pub. No.: US 2011/0160288 A1
(43) Pub. Date: Jun. 30, 2011

(54) OIP5 AS A TARGET GENE FOR CANCER THERAPY AND DIAGNOSIS

(75) Inventors: Yusuke Nakamura, Tokyo (JP); Yataro Daigo, Tokyo (JP); Akira Togashi, Kanagawa (JP)

(73) Assignee: Oncotherapy Science, Inc., Kanagawa (JP)

(21) Appl. No.: 13/061,105

(22) PCT Filed: Aug. 24, 2009
(86) PCT No.: PCT/JP2009/004056

§ 371 Date: Feb. 25, 2011

Related U.S. Application Data
(60) Provisional application No. 61/190,530, filed on Aug. 28, 2008.

(51) Int. Cl. A61K 48/00; C12Q 1/68; G01N 33/574; C07H 21/00; C07K 16/32; C07H 21/04; C12N 15/63; C12Q 1/02; A61P 35/00

(52) U.S. Cl. 514/44 A; 435/6.14; 436/501; 536/24.31; 530/387.7; 536/24.5; 435/320.1; 435/29

(57) ABSTRACT

The present invention relates to the roles played by OIP5 genes in lung and/or esophageal cancer carcinogenesis and features a method for treating and/or preventing lung and/or esophageal cancer by administering a double-stranded molecule against the OIP5 genes or a composition, vector or cell containing such a double-stranded molecule and antibody. The present invention also features methods for detecting and/or diagnosing lung and/or esophageal cancer, or assessing/determining the prognosis of and/or monitoring the efficacy of a cancer therapy in a patient with lung and/or esophageal cancer by detecting OIP5. Also, disclosed are methods of identifying compounds for treating and preventing cancer relating to OIP5.

### PRIORITY

The present application claims the benefit of U.S. Provisional Application No. 61/190,530, filed on Aug. 28, 2008, the entire contents of which are incorporated by reference herein.

### TECHNICAL FIELD

The present invention relates to the field of biological science, more specifically to the field of cancer research, cancer diagnosis and cancer therapy. Moreover, the present invention relates to methods of screening for an agent for treating and/or preventing cancer.

### BACKGROUND ART

Lung cancer is the leading cause of cancer deaths worldwide, and non-small cell lung cancer (NSCLC) accounts for nearly 80% of those cases (Greenlee R T, et al., CA Cancer J Clin 2001; 51: 15-36). Esophageal squamous-cell carcinoma (ESCC) is one of the most lethal malignancies of the digestive tract, and at the time of diagnosis most of the patients are at advanced stages (Shimada H, et al., Surgery 2003; 133: 486-94). In spite of the use of current surgical techniques combined with various treatment modalities, such as radiotherapy and chemotherapy, the overall 5-year survival rate of ESCC still remains at 40% to 60% (Tamoto E, et al., Clin Cancer Res 2004; 10: 3629-38) and that of lung cancer is only 15% (Greenlee R T, et al., CA Cancer J Clin 2001; 51: 15-36).

To isolate potential molecular targets for diagnosis, treatment, and/or prevention of lung and esophageal carcinomas, a genome-wide analysis of gene expression profiles of cancer cells from 101 lung cancer and 19 ESCC patients was performed using a cDNA microarray consisting of 27,648 genes (WO2004/031413, WO2007/013665, WO2007/013671, Daigo Y and Nakamura Y, Gen Thorac Cardiovasc Surg 2008; 56:43-53, Kikuchi T, et al., Oncogene 2003; 22:2192-205, Kakiuchi S, et al., Mol Cancer Res 2003; 1:485-99, Kakiuchi S, et al., Hum Mol Genet 2004; 13:3029-43, Kikuchi T, et al., Int J Oncol 2006; 28:799-805, Taniwaki M, et al., Int J Oncol 2006; 29:567-75, and Yamabuki T, et al., Int J Oncol 2006; 28:1375-84). To verify the biological and clinicopathological significance of the respective gene products, high-throughput screening of loss-of-function effects was performed by means of the RNAi technique and using tumor-tissue microarray analysis of clinical lung-cancer materials (Suzuki C, et al., Cancer Res 2003; 63:7038-41, Ishikawa N, et al., Clin Cancer Res 2004; 10:8363-70, Kato T, et al., Cancer Res 2005; 65:5638-46, Furukawa C, et al., Cancer Res 2005; 65:7102-10, Ishikawa N, et al., Cancer Res 2005; 65:9176-84, Suzuki C, et al., Cancer Res 2005; 65:11314-25, Ishikawa N, et al., Cancer Sci 2006; 97:737-45, Takahashi K, et al. Cancer Res 2006; 66:9408-19, Hayama S, et al., Cancer Res 2006; 66:10339-48, Kato T, et al., Clin Cancer Res 2007; 13:434-42, Suzuki C, et al., Mol Cancer Ther 2007; 6:542-51, Yamabuki T, et al., Cancer Res 2007; 67:2517-25, Hayama S, et al., Cancer Res 2007; 67:4113-22, Kato T, et al., Cancer Res 2007; 67:8544-53, Taniwaki M, et al., Clin Cancer Res 2007; 13:6624-31, Ishikawa N, et al., Cancer Res 2007; 67:11601-11, Mano Y, et al., Cancer Sci 2007; 98:1902-13, Suda T, et al., Cancer Sci 2007; 98:1803-8, Kato T, et al., Clin Cancer Res 2008; 14:2363-70 and Mizukami Y, et al., Cancer Sci 2008; 99:1448-54).

OIP5 (opa interacting protein 5) was found by yeast two-hybrid analysis to interact with Neisseria Gonorrhoeae opacity-associated (Opa) proteins (Williams, J. M., et al., Mol Microbiol 1998; 27(1): 171-86). OIP5 is involved in gonococcal adhesion to and invasion of human epithelial cells. A previous study demonstrated that the elevated expression of OIP5 mRNA in human gastric carcinomas (Nakamura Y, et al., Ann Surg Oncol 2007; 14:885-92.), and, some proteins, such as Raf1, were previously reported to be interacted with OIP5 (Yuryev, A. and L. P. Wennogle, Genomics 2003; 81(2): 112-25), however the biological roles of OIP5 during carcinogenesis have not been clarified.

### CITATION LIST

### Patent Literature

[PTL 1]WO02004/031413
[PTL 2]WO2007/013665
[PTL 3]WO02007/013671

### Non Patent Literature

[NPL 1] Greenlee R T, et al., CA Cancer J Clin 2001; 51: 15-36
[NPL 2] Shimada H, et al., Surgery 2003; 133: 486-94
[NPL 3] Tamoto E, et al., Clin Cancer Res 2004; 10: 3629-38
[NPL 4] Daigo Y and Nakamura Y, Gen Thorac Cardiovasc Surg 2008; 56:43-53
[NPL 5] Kikuchi T, et al., Oncogene 2003; 22:2192-205
[NPL 6] Kakiuchi S, et al., Mol Cancer Res 2003; 1:485-99
[NPL 7] Kakiuchi S, et al., Hum Mol Genet 2004; 13:3029-43
[NPL 8] Kikuchi T, et al., Int J Oncol 2006; 28:799-805
[NPL 9] Taniwaki M, et al., Int J Oncol 2006; 29:567-75
[NPL 10] Yamabuki T, et al., Int J Oncol 2006; 28:1375-84
[NPL 11] Suzuki C, et al., Cancer Res 2003; 63:7038-41
[NPL 12] Ishikawa N, et al., Clin Cancer Res 2004; 10:8363-70
[NPL 13] Kato T, et al., Cancer Res 2005; 65:5638-46
[NPL 14] Furukawa C, et al., Cancer Res 2005; 65:7102-10
[NPL 15] Ishikawa N, et al., Cancer Res 2005; 65:9176-84
[NPL 16] Suzuki C, et al., Cancer Res 2005; 65:11314-25
[NPL 17] Ishikawa N, et al., Cancer Sci 2006; 97:737-45
[NPL 18] Takahashi K, et al. Cancer Res 2006; 66:9408-19
[NPL 19] Hayama S, et al., Cancer Res 2006; 66:10339-48
[NPL 20] Kato T, et al., Clin Cancer Res 2007; 13:434-42
[NPL 21] Suzuki C, et al., Mol Cancer Ther 2007; 6:542-51
[NPL 22] Yamabuki T, et al., Cancer Res 2007; 67:2517-25
[NPL 23] Hayama S, et al., Cancer Res 2007; 67:4113-22
[NPL 24] Kato T, et al., Cancer Res 2007; 67:8544-53
[NPL 25] Taniwaki M, et al., Clin Cancer Res 2007; 13:6624-31
[NPL 26] Ishikawa N, et al., Cancer Res 2007; 67:11601-11
[NPL 27] Mano Y, et al., Cancer Sci 2007; 98:1902-13
[NPL 28] Suda T, et al., Cancer Sci 2007; 98:1803-8
[NPL 29] Kato T, et al., Clin Cancer Res 2008; 14:2363-70
[NPL 30] Mizukami Y, et al., Cancer Sci 2008; 99:1448-54
[NPL 31] Williams, J. M., et al., Mol Microbiol 1998; 27(1): 171-86
[NPL 32] Yuryev, A. and L. P. Wennogle, Genomics 2003; 81(2):
[NPL 33] Nakamura Y, et al., Ann Surg Oncol 2007; 14:885-92.

### SUMMARY OF INVENTION

In this invention, it is disclosed that overexpression of OIP5 can contribute to the malignant nature of lung and esophageal cancer cells. Thus, targeting the OIP5 molecule may hold promise for the development of a new diagnostic and therapeutic strategy in the clinical management of lung and esophageal cancers.

In particular, the present invention arises from the discovery that double-stranded molecules composed of specific sequences (in particular, SEQ ID NOs: 11 and 12) are effective for inhibiting cellular growth of lung and/or esophageal cancer cells. Specifically, small interfering RNAs (siRNAs) targeting OIP5 genes are provided by the present invention. These double-stranded molecules may be utilized in an isolated state or encoded in vectors and expressed from the vectors. Accordingly, it is an object of the present invention to provide such double stranded molecules as well as vectors and host cells expressing them.

In one aspect, the present invention provides methods for inhibiting cell growth and treating lung and/or esophageal cancer by administering the double-stranded molecules or vectors of the present invention to a subject in need thereof. Such methods encompass administering to a subject in need thereof a composition composed of one or more of the double-stranded molecules or vectors of the present invention.

In another aspect, the present invention provides compositions for treating a cancer containing at least one of the double-stranded molecules or vectors of the present invention.

In yet another aspect, the present invention provides a method of diagnosing or determining a predisposition to lung and/or esophageal cancer in a subject by determining an expression level of OIP5 in a patient derived biological sample. An increase in the expression level of the OIP5 gene as compared to a normal control level of the gene indicates that the subject suffers from or is at risk of developing lung and/or esophageal cancer.

Moreover, the present invention relates to the discovery that a high expression level of OIP5 correlates to poor survival rate. Therefore, the present invention provides a method for assessing or determining the prognosis of a patient with lung and/or esophageal cancer, such a method including the steps of detecting the expression level of OIP5, comparing it to a pre-determined reference expression level and determining the prognosis of the patient from the difference there between.

In a further aspect, the present invention provides a method of screening for a compound for treating and/or preventing lung and/or esophageal cancer. Such a compound would bind with the OIP5 polypeptide or reduce the biological activity of the OIP5 polypeptide or reduce the expression of the OIP5 gene or reporter gene surrogating the OIP5 gene or inhibit the binding between the OIP5 polypeptide and the Raf1 polypeptide or inhibit the phosphorylation of the OIP5 polypeptide.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF DRAWINGS

Various aspects and applications of the present invention will become apparent to the skilled artisan upon consideration of the brief description of the figures and the detailed description of the present invention and its preferred embodiments that follows:

FIG. 1 depicts OIP5 expression in lung and esophageal cancers and normal tissues. In Part A, the expression of OIP5 in a normal lung tissue and 15 clinical lung cancer samples [lung adenocarcinoma (ADC), lung SCC, and SCLC; top panels] and 15 lung cancer cell lines (bottom panels) detected by semiquantitative RT-PCR analysis is depicted. In Part B, the expression of OIP5 in a normal esophagus and 10 clinical ESCC tissue samples (top panels) and 10 ESCC cell lines detected by semi-quantitative RT-PCR analysis (bottom panels) is depicted. In Part C, the subcellular localization of endogenous OIP5 protein in SBC-5 cells is depicted. OIP5 was stained in the nucleus and cytoplasm. DAPI, 4′,6-diamidino-2-phenylindole. In Part D, the results of Northern blot analysis of the OIP5 transcript in 16 normal human tissues are depicted. A strong signal was observed in testis.

FIG. 2 depicts OIP5 protein expression and its association with poorer clinical outcomes for NSCLC patients. In Part A, representative examples of expression of OIP5 in lung cancer (squamous cell carcinomas, x100) and normal lung (×100), and magnified view (×200) are depicted. In Part B, the results of Kaplan-Meier analysis of tumor-specific survival in NSCLC patients according to OIP5 expression level (P<0.0099; log-rank test) is depicted.

FIG. 3 depicts the effect of OIP5 on growth of cells. In Part A, the expression of OIP5 in response to si-OIP5s (si-1 and -2) or control siRNAs (LUC and On-Target plus/CNT) in LC319 (left) and SBC5 (right) cells, analyzed by semiquantitative RT-PCR (top panels) is depicted. In particular, viability of LC319 or SBC-5 cells evaluated by MTT assay in response to si-1, si-2, si-LUC, or si-CNT (middle panels). Colony-formation assays of LC319 and SBC-5 cells transfected with specific siRNAs or control siRNAs (bottom panels). All experiments were carried out in triplicate assays. In Part B, the effect of OIP5 on growth of COS-7 cells is depicted. In particular, expression of OIP5 in COS-7 cells examined by western-blot analysis (left top panels). The cells transfected with pCAGGSn3Fc-OIP5 or mock vector were each cultured in triplicate, and the cell viability was evaluated by the MTT assay (right panel). Sizes and numbers of colonies derived from cells transfected with OIP5-expressing plasmids are greater than those with mock vector (left bottom panels).

FIG. 4 depicts the phosphorylation of endogenous and exogenous OIP5. In particular, by treatment with lambda-PPase, upper (phosphorylated) band of endogenous or exogenous OIP5 was diminished.

FIG. 5 depicts OIP5 expression in lung and esophageal cancers and normal tissues. In Part A, the expression of OIP5 in lung cancer cell lines was examined by Western blot analyses. Expression of ACTB was served as a quantity control. In Part B, subcellular localization of endogenous OIP5 protein in SBC-5 cells is depicted. OIP5 was stained in the nucleus and cytoplasm. DAPI indicates 4′,6-diamidino-2-phenylindole.

FIG. 6 depicts OIP5 protein expression in normal tissues and lung and esophageal cancers. In Part A, northern blot analysis of the OIP5 transcript in 23 normal human tissues is depicted. A strong signal was observed in testis. In Part B, the expression of OIP5 in six normal human tissues as well as various histologic types of lung cancers and ESCCs was detected by immunohistochemical staining (Magnification×100). Positive staining appeared predominantly in the nucleus and cytoplasm of the testicular cells and lung cancer cells.

FIG. 7 depits association of OIP5 expression with poorer clinical outcomes for NSCLC and ESCC patients. In Part A, representative examples of OIP5 expression in lung cancer (squamous cell carcinomas) and normal lung (top, X100; bottom X200) are depicted. In Part B, Kaplan-Meier analysis of tumor-specific survival in NSCLC patients according to OIP5 expression level (P=0.0053; log-rank test) is depicted. In Part C, representative examples of OIP5 expression in ESCC and normal esophagus (top, X100; bottom X200) are depicted. In Part D, Kaplan-Meier analysis of tumor-specific survival in ESCC patients according to OIP5 expression level (P=0.0129; log-rank test) is depicted.

FIG. 8 depicts stabilization of OIP5 protein through its interaction with Raf1 protein. In Part A, interaction of exogenous OIP5 with endogenous Raf1 protein in lung cancer cells is depicted. Immunoprecipitations were carried out using M2-Flag agarose and extracts from COS-7 cells that was transfected with pCAGGSn3FC-OIP5-Flag and expressed endogenous Raf1. Immunoprecipitates were subjected to western-blot analysis using anti-Raf1 polyclonal antibody to detect endogenous Raf1. IB indicates immunoblotting; IP indicates immunoprecipitation. In Part B, the expression of OIP5 and Raf1 proteins in lung cancer cell lines is depicted. The expression pattern of OIP5 showed good concordance with that of Raf1 protein. In Part C, effect of Raf1 expression on the levels of OIP5 protein is depicted. In left panels, effect of Raf1 knockdown on the levels of OIP5 protein is depicted. The expression of endogenous Raf1 and OIP5 transcripts as well as their coding proteins was detected by semiquantitative RT-PCR analysis and western-blot analysis in SBC-5 cells transfected with si-Raf1. In right panels, effect of Raf1 overexpression on the levels of OIP5 protein is depicted. The expression levels of Raf1 and OIP5 transcripts and proteins were detected by semiquantitative RT-PCR analysis and western-blot analysis in SBC-5 cells transfected with Raf1 expression vector.

FIG. 9 depicts supplementary figures. In Part A, an antigen blocking assays to examine antibody specificity to OIP5 is depicted. Anti-OIP5 antibody was incubated with recombinant OIP5 protein before immunohistochemical staining (Anti-OIP5+rhOIP5). The positive signal by anti-OIP5 antibody obtained in lung cancer tissues (Anti-OIP5) was diminished by preincubation with rhOIP5. In Part B, enhancement of cellular invasiveness of COS-7 by OIP5 is depicted. Expression of OIP5 in COS-7 cells was examined by western-blot analysis (left top panels). Assays in Matrigel matrix demonstrates the invasive nature of COS-7 cells after transfection with pCAGGSn3Fc-OIP5-Flag. Giemsa staining (bottom panels; magnification, x100), and the relative number of cells migrating through the Matrigel-coated filters (right top panels) were shown. In Part C, direct interaction of OIP5 with Raf1 protein is depicted. Pull-down of OIP5 protein was carried out using anti-His antibody and mixture of His-tagged OIP5 and GST-fused recombinant Raf1 proteins. OIP5-binding Raf1 protein was detected by subsequent western blotting using polyclonal antibody to Raf1 (cell signaling technology). In Part D, the expression of Raf1 in lung cancer cell lines, as detected by semiquantitative RT PCR was depicted.

### DESCRIPTION OF EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

In case of conflict, the present specification, including definitions, will control. In addition, the materials, methods, and examples are illustrative only and not intended to be limiting.

### DEFINITION

The words “a”, “an”, and “the” as used herein mean “at least one” unless otherwise specifically indicated.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

The terms “gene”, “polynucleotide”, “oligonucleotide” “nucleotide”, “nucleic acid”, and “nucleic acid molecule” are used interchangeably herein to refer to a polymer of nucleic acid residues and, unless otherwise specifically indicated are referred to by their commonly accepted single-letter codes. The terms apply to nucleic acid (nucleotide) polymers in which one or more nucleic acids are linked by ester bonding. The nucleic acid polymers may be composed of DNA, RNA or a combination thereof and encompass both naturally-occurring and non-naturally occurring nucleic acid polymers.

The terms “polypeptide”, “peptide”, and “protein” are used interchangeably herein to refer to a polymer of amino acid residues. The terms refer to naturally occurring and synthetic amino acids, as well as amino acids analogs and amino acids mimetics amino acid polymers in which one or more amino acid residue is a modified residue, or a non-naturally occurring residue, such as an artificial chemical mimetic of a corresponding naturally occurring amino acid. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids. Amino acids may be referred to herein by their commonly known three letter symbols or the one-letter symbols recommended by the IUPAC-IUB Biochemical Nomenclature Commission.

Unless otherwise defined, the terms “cancer” refers to cancers over-expressing the OIP5 gene. Examples of cancers over-expressing OIP5 include, but are not limited to, lung and esophageal cancer.

Genes or Proteins

The nucleic acid and polypeptide sequences of genes of interest to the present invention are shown in the following numbers, but not limited to those;

### OIP5: SEQ ID NO: 13 and 14

### Raf1: SEQ ID NO: 17 and 18

Additionally, the sequence datas are available via following accession numbers.

### OIP5: NM_{—}007280;

### Raf1: NM_{—}002880;

According to an aspect of the present invention, functional equivalents are also considered to be above “polypeptides”. Herein, a “functional equivalent” of a protein is a polypeptide that has a biological activity equivalent to the protein. Namely, any polypeptide that retains the biological ability of the original reference peptide may be used as such a functional equivalent in the present invention. Such functional equivalents include those wherein one or more amino acids are substituted, deleted, added, or inserted to the natural occurring amino acid sequence of the protein. Alternatively, the polypeptide may be composed an amino acid sequence having at least about 80% homology (also referred to as sequence identity) to the sequence of the respective protein, more preferably at least about 90% to 95% homology, even more preferably 96% to 99% homology. In other embodiments, the polypeptide can be encoded by a polynucleotide that hybridizes under stringent conditions to the naturally occurring nucleotide sequence of the gene.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of the human protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will vary in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to the above human protein can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

In general. modification of one, two or more amino acid in a protein will not influence the function of the protein. In fact, mutated or modified proteins (i.e., peptides composed of an amino acid sequence in which one, two, or several amino acid residues have been modified through substitution, deletion, insertion and/or addition) have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention. Thus, in one embodiment, the peptides of the present invention may have an amino acid sequence wherein one, two or even more amino acids are added, inserted, deleted, and/or substituted in a reference sequence.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 5 or 6 amino acids or less, and even more preferably 3 or 4 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cystein (C), Methionine (M) (see, e.g., Creighton, Proteins 1984).

Such conservatively modified polypeptides are included in the present protein. However, the present invention is not restricted thereto and includes non-conservative modifications, so long as at least one biological activity of the protein is retained. Furthermore, the modified proteins do not exclude polymorphic variants, interspecies homologues, and those encoded by alleles of these proteins.

Moreover, the gene of the present invention encompasses polynucleotides that encode such functional equivalents of the protein. In addition to hybridization, a gene amplification method, for example, the polymerase chain reaction (PCR) method, can be utilized to isolate a polynucleotide encoding a polypeptide functionally equivalent to the protein, using a primer synthesized based on the sequence above information. Polynucleotides and polypeptides that are functionally equivalent to the human gene and protein, respectively, normally have a high homology to the originating nucleotide or amino acid sequence of. “High homology” typically refers to a homology of 40% or higher, preferably 60% or higher, more preferably 80% or higher, even more preferably 90% to 95% or higher, even more preferably 96% to 99% or higher. The homology of a particular polynucleotide or polypeptide can be determined by following the algorithm in “Wilbur and Lipman, Proc Natl Acad Sci USA 80: 726-30 (1983)”.

Double-Stranded Molecules

As used herein, the term “isolated double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes an OIP5 sense nucleic acid sequence (also referred to as “sense strand”), an OIP5 antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules composed of complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of first and second regions complementary to one another, i.e., sense and antisense strands. The degree of complementarity and orientation of the regions are sufficient such that base pairing occurs between the regions, the first and second regions are joined by a loop region, the loop results from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes an OIP5 sense nucleic acid sequence (also referred to as “sense strand”), an OIP5 antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules composed of complementary sequences to one another and that have annealed together via the complementary sequences to form a double-stranded polynucleotide molecule. The nucleotide sequence of two strands may include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of a first and second regions complementary to one another, i.e., sense and antisense strands. The degree of complementarity and orientation of the regions are sufficient such that base pairing occurs between the regions, the first and second regions are joined by a loop region, the loop results from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the context of the present invention, examples of isolated nucleic acid includes DNA, RNA, and derivatives thereof.

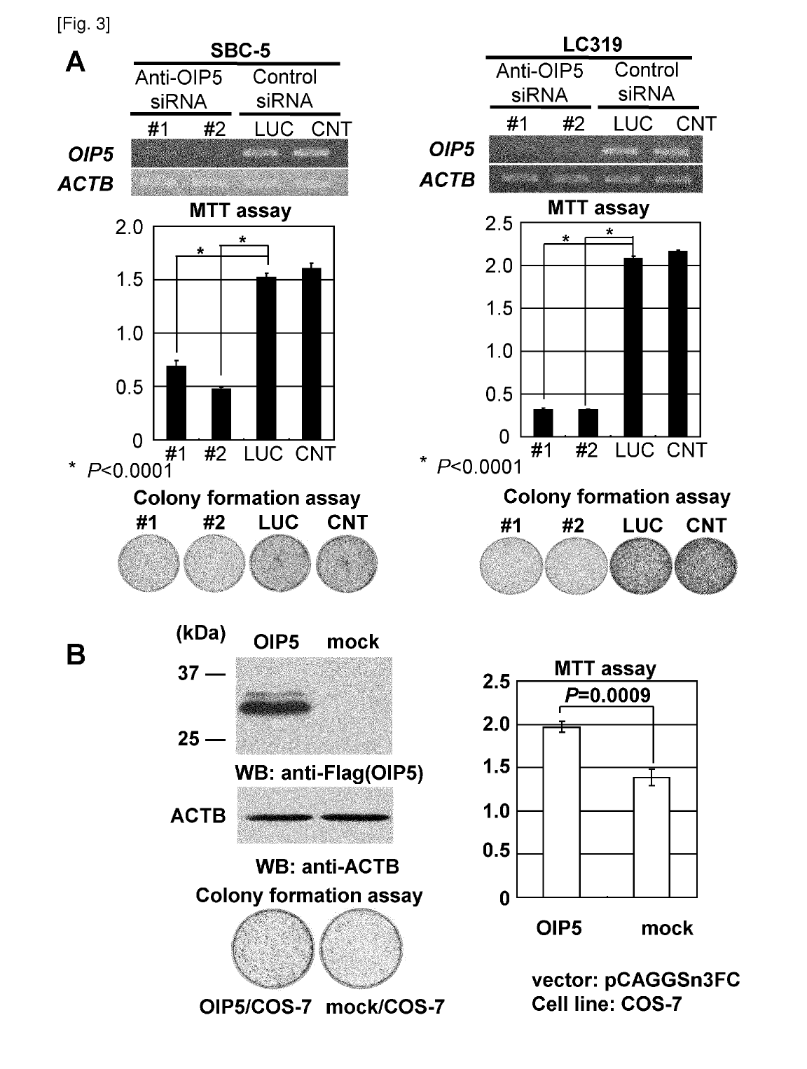

A double-stranded molecule against OIP5 that hybridizes to target mRNA, decreases or inhibits production of OIP5 protein encoded by OIP5 gene by associating with the normally single-stranded mRNA transcript of the gene, thereby interfering with translation and thus, inhibiting expression of the protein. As demonstrated herein, the expression of OIP5 in lung and/or esophageal cancer cell lines was inhibited by dsRNA (FIG. 3A). Accordingly, the present invention provides isolated double-stranded molecules that are capable of inhibiting the expression of an OIP5 gene when introduced into a cell expressing the gene. The target sequence of double-stranded molecules may be designed by an siRNA design algorithm such as that mentioned below.

Examples of OIP5 target sequences include, for example, nucleotides such as:

SEQ ID NO: 11 (at the position 79-97 nt of SEQ ID NO: 13)

SEQ ID NO: 12 (at the position 557-575 nt of SEQ ID NO: 13)

Of particular interest in the present invention are the double-stranded molecules of [1] to [18] set forth below:

[1] An isolated double-stranded molecule that, when introduced into a cell, inhibits in vivo expression of OIP5 and cell proliferation, such molecules composed of a sense strand and an antisense strand complementary thereto, hybridized to each other to form the double-stranded molecule;

[2] The double-stranded molecule of [1], wherein said double-stranded molecule acts on mRNA, matching a target sequence selected from among SEQ ID NOs: 11 (at the position of 79-97 nt of SEQ ID NO: 13), SEQ ID NO: 12 (at the position of 557-575 nt of SEQ ID NO: 13);

[3] The double-stranded molecule of [2], wherein the sense strand contains a sequence corresponding to a target sequence selected from among SEQ ID NOs: 11 and 12;

[4] The double-stranded molecule of [3], having a length of less than about 100 nucleotides;

[5] The double-stranded molecule of [4], having a length of less than about 75 nucleotides;

[6] The double-stranded molecule of [5], having a length of less than about 50 nucleotides;

[7] The double-stranded molecule of [6] having a length of less than about 25 nucleotides;

[8] The double-stranded molecule of [7], having a length of between about 19 and about 25 nucleotides;

[9] The double-stranded molecule of [1], composed of a single polynucleotide having both the sense and antisense strands linked by an intervening single-strand;

[10] The double-stranded molecule of [9], having the general formula 5′-[A]-[B]-[A′]-3′, wherein [A] is the sense strand containing a sequence corresponding to a target sequence selected from among SEQ ID NOs: 11 and 12, [B] is the intervening single-strand composed of 3 to 23 nucleotides, and [A′] is the antisense strand containing a sequence complementary to [A];

[11] The double-stranded molecule of [1], composed of RNA;

[12] The double-stranded molecule of [1], composed of both DNA and RNA;

[13] The double-stranded molecule of [12], wherein the molecule is a hybrid of a DNA polynucleotide and an RNA polynucleotide;

[14] The double-stranded molecule of [13] wherein the sense and the antisense strands are composed of DNA and RNA, respectively;

[15] The double-stranded molecule of [12], wherein the molecule is a chimera of DNA and RNA;

[16] The double-stranded molecule of [15], wherein a region flanking to the 3′-end of the antisense strand, or both of a region flanking to the 5′-end of sense strand and a region flanking to the 3′-end of antisense strand are RNA;

[17] The double-stranded molecule of [16], wherein the flanking region is composed of 9 to 13 nucleotides; and

[18] The double-stranded molecule of [1], wherein the molecule contains 3′ overhang.

The double-stranded molecule of the present invention will be described in more detail below.

Methods for designing double-stranded molecules having the ability to inhibit target gene expression in cells are known. (See, for example, U.S. Pat. No. 6,506,559, herein incorporated by reference in its entirety). For example, a computer program for designing siRNAs is available from the Ambion website (http://www.ambion.com/techlib/misc/siRNA_finder.html).

The computer program selects target nucleotide sequences for double-stranded molecules based on the following protocol.

Selection of Target Sites:

1. Beginning with the AUG start codon of the transcript, scan downstream for AA di-nucleotide sequences. Record the occurrence of each AA and the 3′ adjacent 19 nucleotides as potential siRNA target sites. Tuschl et al. don't recommend designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: www.ncbi.nlm.nih.gov/BLAST/, is used (Altschul S F et al., Nucleic Acids Res 1997 Sep. 1, 25(17): 3389-402).

3. Select qualifying target sequences for synthesis. Selecting several target sequences along the length of the gene to evaluate is typical.

Using the above protocol, the target sequence of the isolated double-stranded molecules of the present invention were designed as:

SEQ ID NO: 11 and 12 for OIP5 gene.

Double-stranded molecules targeting the above-mentioned target sequences were respectively examined for their ability to suppress the growth of cells expressing the target genes. Therefore, the present invention provides double-stranded molecules targeting any of the sequences selected from the group of:

SEQ ID NOs: 11 (at the position 79-97 nt of SEQ ID NO: 13) and 12 (at the position 557-575 nt of SEQ ID NO: 13) for OIP5 gene.

The double-stranded molecule of the present invention may be directed to a single target OIP5 gene sequence or may be directed to a plurality of target OIP5 gene sequences.

A double-stranded molecule of the present invention targeting the above-mentioned targeting sequence of OIP5 gene include isolated polynucleotides that contain any of the nucleic acid sequences of target sequences and/or complementary sequences to the target sequences. Examples of polynucleotides targeting OIP5 gene include those containing the sequence of SEQ ID NO: 11 or 12 and/or complementary sequences to these nucleotides; However, the present invention is not limited to these examples, and minor modifications in the aforementioned nucleic acid sequences are acceptable so long as the modified molecule retains the ability to suppress the expression of OIP5 gene. Herein, the phrase “minor modification” as used in connection with a nucleic acid sequence indicates one, two or several substitution, deletion, addition or insertion of nucleic acids to the sequence.

In the context of the present invention, the term “several” as applies to nucleic acid substitutions, deletions, additions and/or insertions may mean 3-7, preferably 3-5, more preferably 3-4, even more preferably 3 nucleic acid residues.

According to the present invention, a double-stranded molecule of the present invention can be tested for its ability using the methods utilized in the Examples. In the Examples herein below, double-stranded molecules composed of sense strands of various portions of mRNA of OIP5 genes or antisense strands complementary thereto were tested in vitro for their ability to decrease production of an OIP5 gene product in lung and/or esophageal cancer cell lines according to standard methods. Furthermore, for example, reduction in an OIP5 gene product in cells contacted with the candidate double-stranded molecule compared to cells cultured in the absence of the candidate molecule can be detected by, e.g. RT-PCR using primers for OIP5 mRNA mentioned under Example 1 item “Semi-quantitative RT-PCR”. Sequences that decrease the production of an OIP5 gene product in vitro cell-based assays can then be tested for their inhibitory effects on cell growth. Sequences that inhibit cell growth in vitro cell-based assay can then be tested for their in vivo ability using animals with cancer, e.g. nude mouse xenograft models, to confirm decreased production of an OIP5 gene product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is preferably less than 1249 nucleotides in length for OIP5. For example, the polynucleotide is less than 500, 200, 100, 75, 50, or 25 nucleotides in length for all of the genes. The isolated polynucleotides of the present invention are useful for forming double-stranded molecules against OIP5 gene or preparing template DNAs encoding the double-stranded molecules. When the polynucleotides are used for forming double-stranded molecules, the polynucleotide may be longer than 19 nucleotides, preferably longer than 21 nucleotides, and more preferably has a length of between about 19 and 25 nucleotides.

Accordingly, the present invention provides the double-stranded molecules comprising a sense strand and an antisense strand, wherein the sense strand comprises a nucleotide sequence corresponding to a target sequence. In preferable embodiments, the sense strand hybridizes with antisense strand at the target sequence to form the double-stranded molecule having between 19 and 25 nucleotide pair in length.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 January 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the present invention may include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule composed of a DNA strand (polynucleotide) and an RNA strand (polynucleotide), a chimera type double-stranded molecule containing both DNA and RNA on any or both of the single strands (polynucleotides), or the like may be formed for enhancing stability of the double-stranded molecule.

The hybrid of a DNA strand and an RNA strand may be either where the sense strand is DNA and the antisense strand is RNA, or vice versa, so long as it can inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression.

As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention include following combinations.

sense strand:

5′-[---DNA---]-3′

3′-(RNA)-[DNA]-5′

:antisense strand,

sense strand:

5′-(RNA)-[DNA]-3′

3′-(RNA)-[DNA]-5′

:antisense strand, and

sense strand:

5′-(RNA)-[DNA]-3′

3′-(---RNA---)-5′

:antisense strand.

The upstream partial region preferably is a domain composed of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the context of the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand containing a sequence corresponding to a target sequence, [B] is an intervening single-strand and [A′] is the antisense strand containing a complementary sequence to [A]. The target sequence may be selected from among, for example, nucleotides of SEQ ID NOs: 11 and 12 for OIP5.

The present invention is not limited to these examples, and the target sequence in [A] may be modified sequences from these examples so long as the double-stranded molecule retains the ability to suppress the expression of the targeted OIP5 gene. The region [A] hybridizes to [A′] to form a loop composed of the region [B]. The intervening single-stranded portion [B], i.e., loop sequence may be preferably 3 to 23 nucleotides in length. The loop sequence, for example, can be selected from among the following sequences (http://www.ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 June, 4(6): 457-67.

Examples of preferred double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, the present invention is not limited thereto:

(for target sequence SEQ ID NO: 11)
CGGCAUCGCUCACGUUGUG-[B]-CACAACGUGAGCGAUGCCG;
(for target sequence SEQ ID NO: 12)
GUGACAAAAUGGUGUGCUA-[B]-UAGCACACCAUUUUGUCAC;

Furthermore, in order to enhance the inhibition activity of the double-stranded molecules, several nucleotides can be added to 3′end of the sense strand and/or the antisense strand of the target sequence, as 3′ overhangs. The number of nucleotides to be added is at least 2, generally 2 to 10, preferably 2 to 5. The added nucleotides form single strand at the 3′end of the sense strand and/or antisense strand of the double-stranded molecule. The preferred examples of nucleotides to be added include “t” and “u”, but are not limited to. In cases where double-stranded molecules consists of a single polynucleotide to form a hairpin loop structure, a 3′ overhang sequence may be added to the 3′ end of the single polynucleotide.

The method for preparing the double-stranded molecule is not particularly limited though it is preferable to use a chemical synthetic method known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking OIP5 sequences may be identical or different, such that their expression can be modulated independently, or in a temporal or spatial manner. The double-stranded molecules can be transcribed intracellularly by cloning OIP5 gene templates into a vector containing, e.g., a RNA pol III transcription unit from the small nuclear RNA (snRNA) U6 or the human H1 RNA promoter.

Vectors Containing a Double-Stranded Molecule of the Present Invention:

Also included in the present invention are vectors containing one or more of the double-stranded molecules described herein, and a cell containing such a vector.

Of particular interest to the present invention are the vectors of [1] to [10] set forth below:

[1] A vector, encoding a double-stranded molecule that, when introduced into a cell, inhibits in vivo expression of OIP5 and cell proliferation, such molecules composed of a sense strand and an antisense strand complementary thereto, hybridized to each other to form the double-stranded molecule.

[2] The vector of [1], encoding the double-stranded molecule acts on mRNA, matching a target sequence selected from among SEQ ID NO: 11 (at the position of 79-97 nt of SEQ ID NO: 13), SEQ ID NO: 12 (at the position of 557-575 nt of SEQ ID NO: 13);

[3] The vector of [1], wherein the sense strand contains a sequence corresponding to a target sequence selected from among SEQ ID NOs: 11 and 12;

[4] The vector of [3], encoding the double-stranded molecule having a length of less than about 100 nucleotides;

[5] The vector of [4], encoding the double-stranded molecule having a length of less than about 75 nucleotides;

[6] The vector of [5], encoding the double-stranded molecule having a length of less than about 50 nucleotides;

[7] The vector of [6] encoding the double-stranded molecule having a length of less than about 25 nucleotides;

[8] The vector of [7], encoding the double-stranded molecule having a length of between about 19 and about 25 nucleotides;

[9] The vector of [1], wherein the double-stranded molecule is composed of a single polynucleotide having both the sense and antisense strands linked by an intervening single-strand;

[10] The vector of [9], encoding the double-stranded molecule having the general formula 5′-[A]-[B]-[A′]-3′, wherein [A] is the sense strand containing a sequence corresponding to a target sequence selected from among SEQ ID NOs: 11 and 12, [B] is the intervening single-strand composed of 3 to 23 nucleotides, and [A′] is the antisense strand containing a sequence complementary to [A];

A vector of the present invention preferably encodes a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Such vectors of the present invention may be used for producing the present double-stranded molecules, or directly as an active ingredient for treating cancer.

Vectors of the present invention can be produced, for example, by cloning OIP5 sequence into an expression vector so that regulatory sequences are operatively-linked to OIP5 sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti-sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The present invention concerns for the vector including each or both of a combination of polynucleotide including a sense strand nucleic acid and an antisense strand nucleic acid, wherein the antisense strand includes a nucleotide sequence which is complementary to said sense strand, wherein the transcripts of said sense strand and said antisense strand hybridize to each other to form said double-stranded molecule, and wherein said vector, when introduced into a cell expressing the OIP5 gene, inhibits expression of said gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Methods of Inhibiting or Reducing Growth of a Cancer Cell and Treating Cancer Using a Double-Stranded Molecule of the Present Invention:

The ability of certain siRNA to inhibit NSCLC has been previously described in WO 2005/89735, incorporated by reference herein. In present invention, two different dsRNA for OIP5 were tested for their ability to inhibit cell growth. The two dsRNA for OIP5 (FIG. 3A) that effectively knocked down the expression of the gene in lung and esophageal cancer cell lines coincided with suppression of cell proliferation.

Accordingly, the present invention provides methods for inhibiting cell growth, i.e., lung and/or esophageal cancer cell growth, by inducing dysfunction of the OIP5 gene via inhibiting the expression of OIP5. OIP5 gene expression can be inhibited by any of the aforementioned double-stranded molecules of the present invention that specifically target the OIP5 gene or the vectors of the present invention that can express any of the double-stranded molecules.

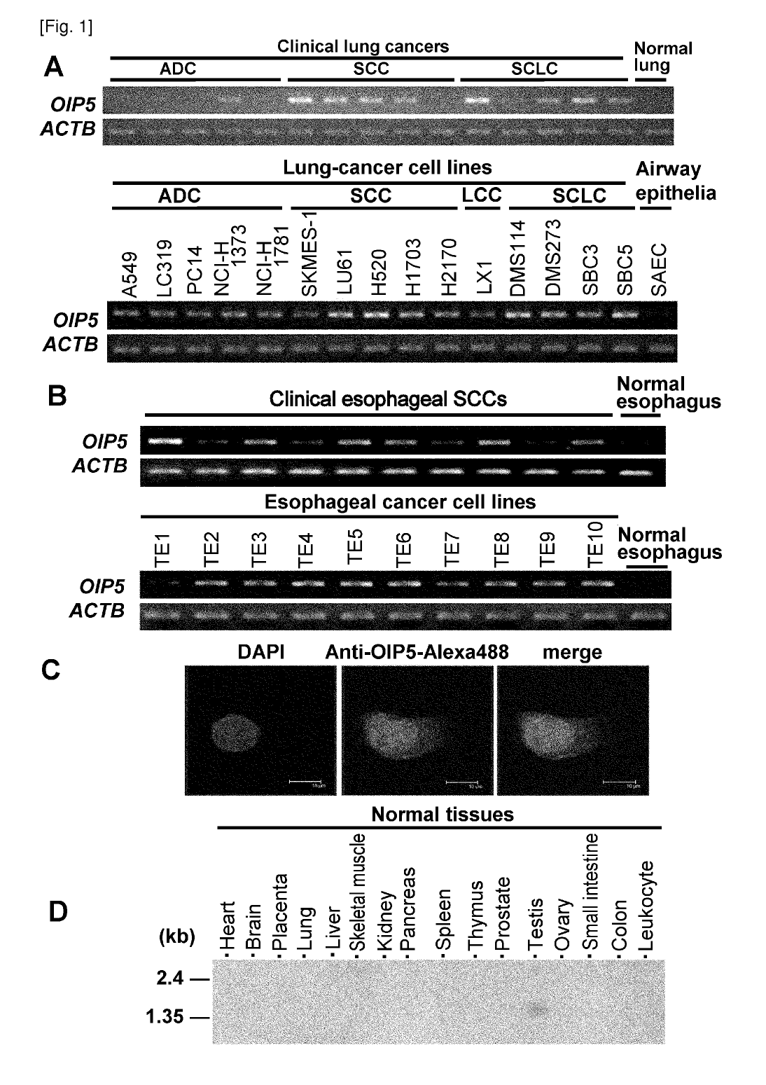

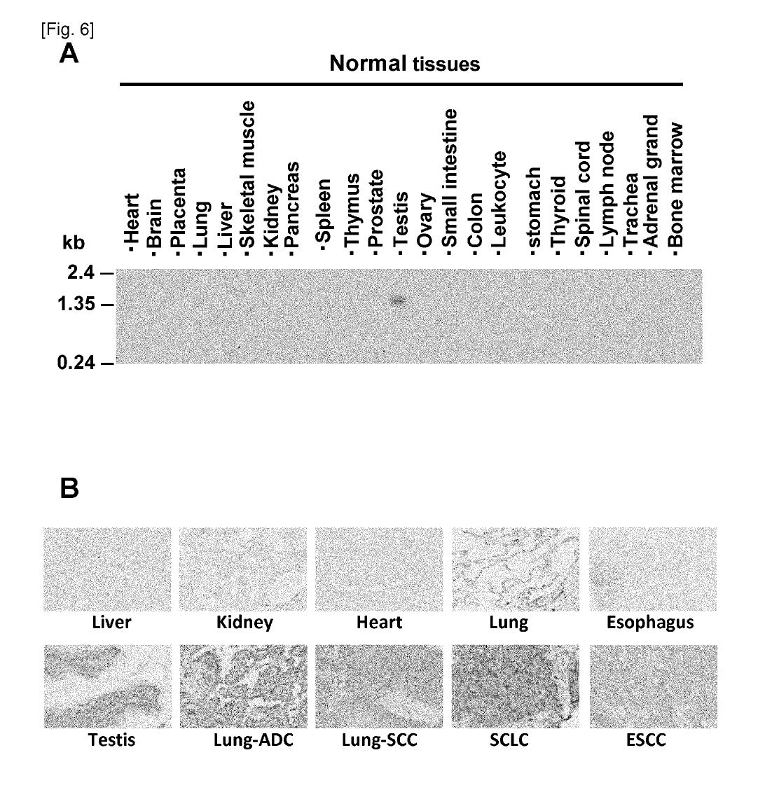

Such ability of the present double-stranded molecules and vectors to inhibit cell growth of cancerous cell indicates that they can be used for methods for treating cancer. Thus, the present invention provides methods to treat patients with cancer by administering a double-stranded molecule against an OIP5 gene or a vector expressing the molecule without adverse effect because that genes were hardly detected in normal organs (FIGS. 1A, B and D, FIG. 6), wherein the cancer is lung and/or esophageal.

Of particular interest to the present invention are the methods of [1] to [36] set forth below:

[1] A method for inhibiting growth of a cancer cell and treating a cancer, wherein the cancer cell or the cancer expresses an OIP5 gene, such method including the step of administering at least one isolated double-stranded molecule inhibiting the expression of OIP5 in a cell over-expressing the gene and the cell proliferation, wherein the double-stranded molecule is composed of a sense strand and an antisense strand complementary thereto, hybridized to each other to form the double-stranded molecule, wherein the sense strand comprises a nucleotide sequence corresponding to a contiguous sequence from SEQ ID NO: 13.

[2] The method of [1], wherein the double-stranded molecule acts at mRNA which matches a target sequence selected from among SEQ ID NO: 11 (at the position of 79-97 nt of SEQ ID NO: 13) and SEQ ID NO: 12 (at the position of 557-575 nt of SEQ ID NO: 13).

[3] The method of [2], wherein the sense strand contains the sequence corresponding to a target sequence selected from among SEQ ID NOs: 11 and 12.

[4] The method of [1], wherein the cancer to be treated is lung and/or esophageal cancer;

[5] The method of [4], wherein the lung cancer is NSCLC or SCLC and esophageal cancer is ESCC;

[6] The method of [1], wherein plural kinds of the double-stranded molecules are administered;

[7] The method of [3], wherein the double-stranded molecule has a length of less than about 100 nucleotides;

[8] The method of [7], wherein the double-stranded molecule has a length of less than about 75 nucleotides;

[9] The method of [8], wherein the double-stranded molecule has a length of less than about 50 nucleotides;

[10] The method of [9], wherein the double-stranded molecule has a length of less than about 25 nucleotides;

[11] The method of [10], wherein the double-stranded molecule has a length of between about 19 and about 25 nucleotides in length;

[12] The method of [1], wherein the double-stranded molecule is composed of a single polynucleotide containing both the sense strand and the antisense strand linked by an intervening single-strand;

[13] The method of [12], wherein the double-stranded molecule has the general formula 5′-[A]-[B]-[A′]-3′, wherein [A] is the sense strand containing a sequence corresponding to a target sequence selected from among SEQ ID NOs: 11 and 12, [B] is the intervening single strand composed of 3 to 23 nucleotides, and [A′] is the antisense strand containing a sequence complementary to [A];

[14] The method of [1], wherein the double-stranded molecule is a RNA;

[15] The method of [1], wherein the double-stranded molecule contains both DNA and RNA;

[16] The method of [15], wherein the double-stranded molecule is a hybrid of a DNA polynucleotide and an RNA polynucleotide;

[17] The method of [16] wherein the sense and antisense strand polynucleotides are composed of DNA and RNA, respectively;

[18] The method of [15], wherein the double-stranded molecule is a chimera of DNA and RNA;

[19] The method of [18], wherein a region flanking to the 3′-end of the antisense strand, or both of a region flanking to the 5′-end of sense strand and a region flanking to the 3′-end of antisense strand are composed of RNA;

[20] The method of [19], wherein the flanking region is composed of 9 to 13 nucleotides;

[21] The method of [1], wherein the double-stranded molecule contains 3′ overhangs;

[22] The method of [1], wherein the double-stranded molecule is contained in a composition which includes, in addition to the molecule, a transfection-enhancing agent and pharmaceutically acceptable carrier.

[23] The method of [1], wherein the double-stranded molecule is encoded by a vector;

[24] The method of [23], wherein the double-stranded molecule encoded by the vector acts at mRNA which matches a target sequence selected from among SEQ ID NO: 11 (at the position of 79-97 nt of SEQ ID NO: 13) and SEQ ID NO: 12 (at the position of 557-575 nt of SEQ ID NO: 13).

[25] The method of [24], wherein the sense strand of the double-stranded molecule encoded by the vector contains the sequence corresponding to a target sequence selected from among SEQ ID NOs: 11 and 12.

[26] The method of [23], wherein the cancer to be treated is lung and/or esophageal cancer;

[27] The method of [26], wherein the lung cancer is NSCLC or SCLC and esophageal cancer is ESCC;

[28] The method of [23], wherein plural kinds of the double-stranded molecules are administered;

[29] The method of [25], wherein the double-stranded molecule encoded by the vector has a length of less than about 100 nucleotides;

[30] The method of [29], wherein the double-stranded molecule encoded by the vector has a length of less than about 75 nucleotides;

[31] The method of [30], wherein the double-stranded molecule encoded by the vector has a length of less than about 50 nucleotides;

[32] The method of [31], wherein the double-stranded molecule encoded by the vector has a length of less than about 25 nucleotides;

[33] The method of [32], wherein the double-stranded molecule encoded by the vector has a length of between about 19 and about 25 nucleotides in length;

[34] The method of [23], wherein the double-stranded molecule encoded by the vector is composed of a single polynucleotide containing both the sense strand and the antisense strand linked by an intervening single-strand;

[35] The method of [34], wherein the double-stranded molecule encoded by the vector has the general formula 5′-[A]-[B]-[A′]-3′, wherein [A] is the sense strand containing a sequence corresponding to a target sequence selected from among SEQ ID NOs: 11 and 12, [B] is a intervening single-strand is composed of 3 to 23 nucleotides, and [A′] is the antisense strand containing a sequence complementary to [A]; and

[36] The method of [23], wherein the double-stranded molecule encoded by the vector is contained in a composition which includes, in addition to the molecule, a transfection-enhancing agent and pharmaceutically acceptable carrier.

The method of the present invention will be described in more detail below.

The growth of cells expressing an OIP5 gene may be inhibited by contacting the cells with a double-stranded molecule against an OIP5 gene, a vector expressing the molecule or a composition containing the same. The cell may be further contacted with a transfection agent. Suitable transfection agents are known in the art. The phrase “inhibition of cell growth” indicates that the cell proliferates at a lower rate or has decreased viability as compared to a cell not exposed to the molecule. Cell growth may be measured by methods known in the art, e.g., using the MTT cell proliferation assay.

The growth of any kind of cell may be suppressed according to the present method so long as the cell expresses or over-expresses the target gene of the double-stranded molecule of the present invention. Exemplary cells include lung and/or esophageal cancer cells, particularly NSCLC, SCLC and ESCC.

Thus, patients suffering from or at risk of developing disease related to OIP5 may be treated with the administration of at least one of the present double-stranded molecules, at least one vector expressing at least one of the molecules or at least one composition containing at least one of the molecules. For example, patients suffering from lung and/or esophageal cancer may be treated according to the present methods. The type of cancer may be identified by standard methods according to the particular type of tumor to be diagnosed. Lung and/or esophageal cancer may be diagnosed, for example, with Carcinoembryonic antigen (CEA), CYFRA, pro-GRP and so on, as lung and/or esophageal cancer marker, or with Chest X-Ray and/or Sputum Cytology. More preferably, patients treated by the methods of the present invention are selected by detecting the expression of OIP5 in a biopsy from the patient by RT-PCR or immunoassay. Preferably, before the treatment of the present invention, the biopsy specimen from the subject is confirmed for OIP5 gene over-expression by methods known in the art, for example, immunohistochemical analysis or RT-PCR.

According to the present method, to inhibit cell growth and thereby treat cancer through the administration of plural kinds of the double-stranded molecules (or vectors expressing or compositions containing the same), each of the molecules may have different structures but act on an mRNA that matches the same target sequence of OIP5. Alternatively, plural kinds of double-stranded molecules may act on an mRNA that matches a different target sequence of same gene. For example, the method may utilize double-stranded molecules directed to OIP5.

For inhibiting cell growth, a double-stranded molecule of present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Roche diagnostics), Lipofectamine 2000 (Invitrogen), Oligofectamine (Invitrogen), and Nucleofector (Wako pure Chemical), may be employed.

A treatment is deemed “efficacious” if it leads to clinical benefit such as, reduction in expression of OIP5 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

To the extent that the methods and compositions of the present invention find utility in the context of “prevention” and “prophylaxis”, such terms are interchangeably used herein to refer to any activity that reduces the burden of mortality or morbidity from disease. Prevention and prophylaxis can occur “at primary, secondary and tertiary prevention levels.” While primary prevention and prophylaxis avoid the development of a disease, secondary and tertiary levels of prevention and prophylaxis encompass activities aimed at the prevention and prophylaxis of the progression of a disease and the emergence of symptoms as well as reducing the negative impact of an already established disease by restoring function and reducing disease-related complications. Alternatively, prevention and prophylaxis can include a wide range of prophylactic therapies aimed at alleviating the severity of the particular disorder, e.g. reducing the proliferation and metastasis of tumors.

The treatment and/or prophylaxis of cancer and/or the prevention of postoperative recurrence thereof include any of the following steps, such as the surgical removal of cancer cells, the inhibition of the growth of cancerous cells, the involution or regression of a tumor, the induction of remission and suppression of occurrence of cancer, the tumor regression, and the reduction or inhibition of metastasis. Effectively treating and/or the prophylaxis of cancer decreases mortality and improves the prognosis of individuals having cancer, decreases the levels of tumor markers in the blood, and alleviates detectable symptoms accompanying cancer. For example, reduction or improvement of symptoms constitutes effectively treating and/or the prophylaxis include 10%, 20%, 30% or more reduction, or stable disease.

It is understood that a double-stranded molecule of the invention degrades OIP5 mRNA in substoichiometric amounts. Without wishing to be bound by any theory, it is believed that the double-stranded molecule of the invention causes degradation of the target mRNA in a catalytic manner. Thus, as compared to standard cancer therapies, the present invention requires the delivery of significantly less double-stranded molecule at or near the site of cancer in order to exert therapeutic effect.

One skilled in the art can readily determine an effective amount of the double-stranded molecule of the invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the invention is an intercellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered. The precise dosage required for a particular circumstance may be readily and routinely determined by one of skill in the art.

The present methods can be used to inhibit the growth or metastasis of cancer expressing at least one OIP5; for example, lung and/or esophageal cancer, especially NSCLC, SCLC or ESCC. In particular, a double-stranded molecule containing a target sequence of OIP5 (i.e., SEQ ID NOs: 11 or 12) is particularly preferred for the treatment of lung and/or esophageal cancer.

For treating cancer, the double-stranded molecule of the invention can also be administered to a subject in combination with a pharmaceutical agent different from the double-stranded molecule. Alternatively, the double-stranded molecule of the invention can be administered to a subject in combination with another therapeutic method designed to treat cancer. For example, the double-stranded molecule of the invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present a double-stranded molecule include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as lung and/or esophageal tumor tissue, and can also increase the blood half-life of the double-stranded molecule. Liposomes suitable for use in the context of the present invention may be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecule includes a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can include both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as poly-acrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH. sub. 3 and a solvent mixture such as tetrahydrofuran and water in a 30:12 ratio at 60 degrees C.

Vectors expressing a double-stranded molecule of the present invention are discussed above. Such vectors expressing at least one double-stranded molecule of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravesical or intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or deposition including subcutaneous infusion (such as by osmotic pumps); direct application to the area at or near the site of cancer, for example by a catheter or other placement device (e.g., a suppository or an implant including a porous, non-porous, or gelatinous material); and inhalation. It is preferred that injections or infusions of the double-stranded molecule or vector be given at or near the site of the cancer.

The double-stranded molecule of the invention can be administered in a single dose or in multiple doses. Where the administration of the double-stranded molecule of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen includes multiple administrations, it is understood that the effective amount of a double-stranded molecule administered to the subject can include the total amount of a double-stranded molecule administered over the entire dosage regimen.

Compositions Containing a Double-Stranded Molecule of the Present Invention:

In addition to the above, the present invention also provides pharmaceutical compositions that include at least one of the present double-stranded molecules or the vectors coding for the molecules. Of particular interest to the present invention are the following compositions [1] to [36]:

[1] A composition for inhibiting a growth of a cancer cell and treating a cancer, wherein the cancer and the cancer cell express at least one OIP5 gene, including at least one isolated double-stranded molecule that inhibits the expression of OIP5 and the cell proliferation, further wherein molecule is composed of a sense strand and an antisense strand complementary thereto, hybridized to each other to form the double-stranded molecule.

[2] The composition of [1], wherein the double-stranded molecule acts at mRNA which matches a target sequence selected from among SEQ ID NO: 11 (at the position of 79-97 nt of SEQ ID NO: 13) and SEQ ID NO:12 (at the position of 557-575 nt of SEQ ID NO: 13).

[3] The composition of [2], wherein the double-stranded molecule, wherein the sense strand contains a sequence corresponding to a target sequence selected from among SEQ ID NOs: 11 and 12.

[4] The composition of [1], wherein the cancer to be treated is lung and/or esophageal cancer;

[5] The composition of [4], wherein the lung cancer is NSCLC or SCLC and esophageal cancer is ESCC;

[6] The composition of [1], wherein the composition contains plural kinds of the double-stranded molecules;

[7] The composition of [3], wherein the double-stranded molecule has a length of less than about 100 nucleotides;

[8] The composition of [7], wherein the double-stranded molecule has a length of less than about 75 nucleotides;

[9] The composition of [8], wherein the double-stranded molecule has a length of less than about 50 nucleotides;

[10] The composition of [9], wherein the double-stranded molecule has a length of less than about 25 nucleotides;

[11] The composition of [10], wherein the double-stranded molecule has a length of between about 19 and about 25 nucleotides;

[12] The composition of [1], wherein the double-stranded molecule is composed of a single polynucleotide containing the sense strand and the antisense strand linked by an intervening single-strand;

[13] The composition of [12], wherein the double-stranded molecule has the general formula 5′-[A]-[B]-[A′]-3′, wherein [A] is the sense strand sequence contains a sequence corresponding to a target sequence selected from among SEQ ID NOs: 11 and 12, [B] is the intervening single-strand consisting of 3 to 23 nucleotides, and [A′] is the antisense strand contains a sequence complementary to [A];

[14] The composition of [1], wherein the double-stranded molecule is an RNA;

[15] The composition of [1], wherein the double-stranded molecule is DNA and/or RNA;

[16] The composition of [15], wherein the double-stranded molecule is a hybrid of a DNA polynucleotide and an RNA polynucleotide;

[17] The composition of [16], wherein the sense and antisense strand polynucleotides are composed of DNA and RNA, respectively;

[18] The composition of [15], wherein the double-stranded molecule is a chimera of DNA and RNA;

[19] The composition of [18], wherein a region flanking to the 3′-end of the antisense strand, or both of a region flanking to the 5′-end of sense strand and a region flanking to the 3′-end of antisense strand are composed of RNA;

[20] The composition of [19], wherein the flanking region is composed of 9 to 13 nucleotides;

[21] The composition of [1], wherein the double-stranded molecule contains 3′ overhangs;

[22] The composition of [1], wherein the composition includes a transfection-enhancing agent and pharmaceutically acceptable carrier.

[23] The composition of [1], wherein the double-stranded molecule is encoded by a vector and contained in the composition;

[24] The composition of [23], wherein the double-stranded molecule encoded by the vector acts at mRNA which matches a target sequence selected from among SEQ ID NO: 11 (at the position of 79-97 nt of SEQ ID NO: 13), and SEQ ID NO: 12 (at the position of 557-575 nt of SEQ ID NO: 13).

[25] The composition of [24], wherein the sense strand of the double-stranded molecule encoded by the vector contains the sequence corresponding to a target sequence selected from among SEQ ID NOs: 11 and 12.

[26] The composition of [23], wherein the cancer to be treated is lung and/or esophageal cancer;

[27] The composition of [26], wherein the lung cancer is NSCLC or SCLC and esophageal cancer is ESCC;

[28] The composition of [23], wherein plural kinds of the double-stranded molecules are administered;

[29] The composition of [25], wherein the double-stranded molecule encoded by the vector has a length of less than about 100 nucleotides;

[30] The composition of [29], wherein the double-stranded molecule encoded by the vector has a length of less than about 75 nucleotides;

[31] The composition of [30], wherein the double-stranded molecule encoded by the vector has a length of less than about 50 nucleotides;

[32] The composition of [31], wherein the double-stranded molecule encoded by the vector has a length of less than about 25 nucleotides;

[33] The composition of [32], wherein the double-stranded molecule encoded by the vector has a length of between about 19 and about 25 nucleotides in length;

[34] The composition of [23], wherein the double-stranded molecule encoded by the vector is composed of a single polynucleotide containing both the sense strand and the antisense strand linked by an intervening single-strand;

[35] The composition of [23], wherein the double-stranded molecule has the general formula 5′-[A]-[B]-[A′]-3′, wherein [A] is the sense strand containing a sequence corresponding to a target sequence selected from among SEQ ID NOs: 11 and 12, [B] is a intervening single-strand composed of 3 to 23 nucleotides, and [A′] is the antisense strand containing a sequence complementary to [A]; and

[36] The composition of [23], wherein the composition includes a transfection-enhancing agent and pharmaceutically acceptable carrier.

Suitable compositions of the present invention are described in additional detail below.

The double-stranded molecules of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations contain at least one of the double-stranded molecules or vectors encoding them of the present invention (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain plural kinds of the double-stranded molecules, each of the molecules may be directed to the same target sequence, or different target sequences of OIP5. For example, the composition may contain double-stranded molecules directed to OIP5. Alternatively, for example, the composition may contain double-stranded molecules directed to one, two or more target sequences OIP5.

Furthermore, the present composition may contain a vector coding for one or plural double-stranded molecules. For example, the vector may encode one, two or several kinds of the present double-stranded molecules. Alternatively, the present composition may contain plural kinds of vectors, each of the vectors coding for a different double-stranded molecule.

Moreover, the present double-stranded molecules may be contained as liposomes in the present composition. See under the item of “Methods of treating cancer using the double-stranded molecule” for details of liposomes.

Pharmaceutical compositions of the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25-75%, of one or more double-stranded molecule of the invention. A pharmaceutical composition for aerosol (inhalational) administration can include 0.01-20% by weight, preferably 1-10% by weight, of one or more double-stranded molecule of the invention encapsulated in a liposome as described above, and propellant. A carrier can also be included as desired; e.g., lecithin for intranasal delivery.

In addition to the above, the present composition may contain other pharmaceutically active ingredients, so long as they do not inhibit the in vivo function of the double-stranded molecules of the present invention. For example, the composition may contain chemotherapeutic agents conventionally used for treating cancers.

In another embodiment, the present invention provides for the use of the double-stranded nucleic acid molecules of the present invention in manufacturing a pharmaceutical composition for use in treating a lung and/or esophageal cancer characterized by the expression of OIP5. For example, the present invention relates to a use of double-stranded nucleic acid molecule inhibiting the expression of an OIP5 gene in a cell, which molecule includes a sense strand and an antisense strand complementary thereto, hybridized to each other to form the double-stranded nucleic acid molecule and targets to a sequence selected from among SEQ ID NOs: 11 and 12, for manufacturing a pharmaceutical composition for use in treating lung and/or esophageal cancer expressing OIP5.

The present invention further provides a method or process for manufacturing a pharmaceutical composition for treating a lung and/or esophageal cancer characterized by the expression of OIP5, wherein the method or process includes a step for formulating a pharmaceutically or physiologically acceptable carrier with a double-stranded nucleic acid molecule inhibiting the expression of OIP5 in a cell, which over-expresses the gene, which molecule includes a sense strand and an antisense strand complementary thereto, hybridized to each other to form the double-stranded nucleic acid molecule and targets to a sequence selected from among SEQ ID NOs: 11 and 12 as active ingredients.

In another embodiment, the present invention provides a method or process for manufacturing a pharmaceutical composition for treating a lung and/or esophageal cancer characterized by the expression of OIP5, wherein the method or process includes a step for admixing an active ingredient with a pharmaceutically or physiologically acceptable carrier, wherein the active ingredient is a double-stranded nucleic acid molecule inhibiting the expression of OIP5 in a cell, which over-expresses the gene, which molecule includes a sense strand and an antisense strand complementary thereto, hybridized to each other to form the double-stranded nucleic acid molecule and targets to a sequence selected from among SEQ ID NOs: 11 and 12.

Method of Detecting or Diagnosing Lung and/or Esophageal Cancer:

The expression of OIP5 was found to be specifically elevated in lung and/or esophageal cancer cells (FIGS. 1A and B). Accordingly, the genes identified herein as well as their transcription and translation products find diagnostic utility as markers for lung and/or esophageal cancer and by measuring the expression of OIP5 in a cell sample, lung and/or esophageal cancer can be diagnosed. Specifically, the present invention provides a method for detecting, diagnosing and/or determining the presence of or a predisposition for developing lung and/or esophageal cancer by determining the expression level of OIP5 in the subject. Lung and/or esophageal cancers that can be diagnosed by the present method include NSCLC, SCLC and ESCC. Furthermore, NSCLC, including lung and/or esophageal adenocarcinoma and lung and/or esophageal squamous cell carcinoma (SCC), can also be diagnosed or detected by the present invention.

According to the present invention, an intermediate result for examining the condition of a subject may be provided. Such intermediate result may be combined with additional information to assist a doctor, nurse, or other practitioner to determine that a subject suffers from the disease. That is, the present invention provides a diagnostic marker OIP5 for examining cancer.

Alternatively, the present invention provides a method for detecting or identifying cancer cells in a subject-derived lung or esophageal tissue sample, said method including the step of determining the expression level of the OIP5 gene in a subject-derived biological sample, wherein an increase in said expression level as compared to a normal control level of said gene indicates the presence or suspicion of cancer cells in the tissue.

Such result may be combined with additional information to assist a doctor, nurse, or other healthcare practitioner in diagnosing a subject as afflicted with the disease. In other words, the present invention may provide a doctor with useful information to diagnose a subject as afflicted with the disease. For example, according to the present invention, when there is doubt regarding the presence of cancer cells in the tissue obtained from a subject, clinical decisions can be reached by considering the expression level of the OIP5 gene, plus a different aspect of the disease including tissue pathology, levels of known tumor marker(s) in blood, and clinical course of the subject, etc. For example, some well-known diagnostic lung tumor markers in blood are IAP, ACT, BFP, CA19-9, CA50, CA72-4, CA130, CEA, KMO-1, NSE, SCC, SP1, Span-1, TPA, CSLEX, SLX, STN and CYFRA. Alternatively, diagnostic esophageal tumor markers in blood such as CEA, DUPAN-2, IAP, NSE, SCC, SLX and Span-1 are also well known. Namely, in this particular embodiment of the present invention, the outcome of the gene expression analysis serves as an intermediate result for further diagnosis of a subject's disease state.

Of particular interest to the present invention are the following methods [1] to [10]:

[1] A method for diagnosing lung and/or esophageal cancer, said method including the steps of:

(a) detecting the expression level of the gene encoding the amino acid sequence of OIP5 in a biological sample; and

(b) correlating an increase in the expression level detected as compared to a normal control level of the gene to the presence of disease.

[2] The method of [1], wherein the expression level is at least 10% greater than the normal control level.

[3] The method of [1], wherein the expression level is detected by a method selected from among:

(a) detecting an mRNA including the sequence of OIP5,

(b) detecting a protein including the amino acid sequence of OIP5, and

(c) detecting a biological activity of a protein including the amino acid sequence of OIP5.

[4] The method of [1], wherein the lung cancer is NSCLC or SCLC, and the esophageal cancer is ESCC.

[5] The method of [3], wherein the expression level is determined by detecting hybridization of a probe to a gene transcript of the gene.

[6] The method of [3], wherein the expression level is determined by detecting the binding of an antibody against the protein encoded by a gene as the expression level of the gene.

[7] The method of [1], wherein the biological sample includes biopsy, sputum or blood.

[8] The method of [1], wherein the subject-derived biological sample includes an epithelial cell.

[9] The method of [1], wherein the subject-derived biological sample includes a cancer cell.

[10] The method of [1], wherein the subject-derived biological sample includes a cancerous epithelial cell.

The method of diagnosing lung and/or esophageal cancer will be described in more detail below.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of OIP5. The biological samples include, but are not limited to, bodily tissues which are desired for diagnosing or are suspicion of suffering from cancer, and fluids, such as biopsy, blood, sputum, pleural effusion and urine. Preferably, the biological sample contains a cell population including an epithelial cell, more preferably a cancerous epithelial cell or an epithelial cell derived from tissue suspected to be cancerous. Further, if necessary, the cell may be purified from the obtained bodily tissues and fluids, and then used as the biological sample.

According to the present invention, the expression level of OIP5 in the subject-derived biological sample is determined. The expression level can be determined at the transcription (nucleic acid) product level, using methods known in the art. For example, the mRNA of OIP5 may be quantified using probes by hybridization methods (e.g., Northern hybridization). The detection may be carried out on a chip or an array. The use of an array is preferable for detecting the expression level of a plurality of genes (e.g., various cancer specific genes) including OIP5. Those skilled in the art can prepare such probes utilizing the sequence information of the OIP5 (SEQ ID NO 13; GenBank accession number: NM_{—}007280). For example, the cDNA of OIP5 may be used as the probes. If necessary, the probe may be labeled with a suitable label, such as dyes, fluorescent and isotopes, and the expression level of the gene may be detected as the intensity of the hybridized labels.

Furthermore, the transcription product of OIP5 may be quantified using primers by amplification-based detection methods (e.g., RT-PCR). Such primers can also be prepared based on the available sequence information of the gene. For example, the primer pairs (SEQ ID NOs: 1 and 2, or 5 and 6) used in the Example may be employed for the detection by RT-PCR or Northern blot, but the present invention is not restricted thereto.

Specifically, a probe or primer used for the present method hybridizes under stringent, moderately stringent, or low stringent conditions to the mRNA of OIP5. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

Alternatively, the translation product may be detected for the diagnosis of the present invention. For example, the quantity of OIP5 protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to OIP5 protein. Methods to prepare these kinds of antibodies for the detection of proteins are well known in the art, and any method may be employed in the present invention to prepare such antibodies and equivalents thereof.

As another method to detect the expression level of OIP5 gene based on its translation product, the intensity of staining may be observed via immunohistochemical analysis using an antibody against OIP5 protein. Namely, the observation of strong staining indicates increased presence of the protein and at the same time high expression level of OIP5 gene.

Moreover, in addition to the expression level of OIP5 gene, the expression level of other cancer-associated genes, for example, genes known to be differentially expressed in lung and/or esophageal cancer may also be determined to improve the accuracy of the diagnosis.

The expression level of cancer marker gene including OIP5 gene in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of OIP5 gene in samples from subjects whose disease state are known. Furthermore, the control level can be a database of expression patterns from previously tested cells. Moreover, according to an aspect of the present invention, the expression level of OIP5 gene in a biological sample may be compared to multiple control levels, which control levels are determined from multiple reference samples. It is preferred to use a control level determined from a reference sample derived from a tissue type similar to that of the patient-derived biological sample. Moreover, it is preferred, to use the standard value of the expression levels of OIP5 gene in a population with a known disease state. The standard value may be obtained by any method known in the art. For example, a range of mean+/−2 S.D. or mean+/−3 S.D. may be used as standard value.

In the context of the present invention, a control level determined from a biological sample that is known to be non-cancerous is referred to as a “normal control level”. On the other hand, if the control level is determined from a cancerous biological sample, it is referred to as a “cancerous control level”.

When the expression level of OIP5 gene is increased as compared to the normal control level or is similar to the cancerous control level, the subject may be diagnosed to be suffering from or at a risk of developing cancer. Furthermore, in the case where the expression levels of multiple cancer-related genes are compared, a similarity in the gene expression pattern between the sample and the reference that is cancerous indicates that the subject is suffering from or at a risk of developing cancer.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

Method for Assessing the Prognosis of Cancer:

The present invention relates to the novel discovery that OIP5 expression is significantly associated with poorer prognosis of patients. Thus, the present invention provides a method for determining or assessing the prognosis of a patient with cancer, in particular lung and/or esophageal cancer, by detecting the expression level of the OIP5 in a biological sample of the patient; comparing the detected expression level to a control level; and determining a increased expression level to the control level as indicative of poor prognosis (poor survival).

In addition, the expression level of the OIP5 gene before and after a treatment can be compared according to the present method to assess the efficacy of the treatment. Specifically, the expression level detected in a subject-derived biological sample after a treatment (i.e., post-treatment level) is compared to the expression level detected in a biological sample obtained prior to treatment onset from the same subject (i.e., pre-treatment level). A decrease in the post-treatment level compared to the pre-treatment level indicates that the treatment of interest is efficacious while an increase in or similarity of the post-treatment level to the pre-treatment level indicates less favorable clinical outcome or prognosis.

As used herein, the term “efficacious” indicates that the treatment leads to a reduction in the expression of a pathologically up-regulated gene, an increase in the expression of a pathologically down-regulated gene or a decrease in size, prevalence, or metastatic potential of carcinoma in a subject. When a treatment of interest is applied prophylactically, “efficacious” means that the treatment retards or prevents the forming of tumor or retards, prevents, or alleviates at least one clinical symptom of the disease. Assessment of the state of tumor in a subject can be made using standard clinical protocols.

In addition, efficaciousness of a treatment can be determined in association with any known method for diagnosing cancer. Cancers can be diagnosed, for example, by identifying symptomatic anomalies, e.g., weight loss, abdominal pain, back pain, anorexia, nausea, vomiting and generalized malaise, weakness, and jaundice.

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

The terms “assessing the prognosis” refer to the ability of predicting, forecasting or correlating a given detection or measurement with a future outcome of cancer of the patient (e.g., malignancy, likelihood of curing cancer, survival, and the like). For example, a determination of the expression level of OIP5 over time enables a predicting of an outcome for the patient (e.g., increase or decrease in malignancy, increase or decrease in grade of a cancer, likelihood of curing cancer, survival, and the like).

In the context of the present invention, the phrase “assessing (or determining) the prognosis” is intended to encompass predictions and likelihood analysis of cancer, progression, particularly cancer recurrence, metastatic spread and disease relapse. The present method for assessing prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the OIP5 gene can be detected in the sample. Preferably, the biological sample is a lung and/or esophageal cell (a cell obtained from the lung and/or esophageal). Furthermore, the biological sample may include bodily fluids such as sputum, blood, serum, or plasma. Moreover, the sample may be cells purified from a tissue. The biological samples may be obtained from a patient at various time points, including before, during, and/or after a treatment.

According to the present invention, the higher the expression level of the OIP5 gene measured in the patient-derived biological sample, the poorer the prognosis for post-treatment remission, recovery, and/or survival and the higher the likelihood of poor clinical outcome. Thus, according to the present method, the “control level” used for comparison may be, for example, the expression level of the OIP5 gene detected before any kind of treatment in an individual or a population of individuals who showed good or positive prognosis of cancer, after the treatment, which herein will be referred to as “good prognosis control level”. Alternatively, the “control level” may be the expression level of the OIP5 gene detected before any kind of treatment in an individual or a population of individuals who showed poor or negative prognosis of cancer, after the treatment, which herein will be referred to as “poor prognosis control level”. The “control level” is a single expression pattern derived from a single reference population or from a plurality of expression patterns. Thus, the control level may be determined based on the expression level of the OIP5 gene detected before any kind of treatment in a patient of cancer, or a population of the patients whose disease state (good or poor prognosis) is known. Preferably, cancer is lung and/or esophageal cancer. It is preferred, to use the standard value of the expression levels of the OIP5 gene in a patient group with a known disease state. The standard value may be obtained by any method known in the art. For example, a range of mean+/−2 S.D. or mean+/−3 S.D. may be used as standard value.

The control level may be determined at the same time with the test biological sample by using a sample(s) previously collected and stored before any kind of treatment from cancer patient(s) (control or control group) whose disease state (good prognosis or poor prognosis) are known.

Alternatively, the control level may be determined by a statistical method based on the results obtained by analyzing the expression level of the OIP5 gene in samples previously collected and stored from a control group. Furthermore, the control level can be a database of expression patterns from previously tested cells.

Moreover, according to an aspect of the present invention, the expression level of the OIP5 gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the OIP5 gene to a good prognosis control level indicates a more favorable prognosis of the patient and an increase in the expression level to the good prognosis control level indicates less favorable, poorer prognosis for post-treatment remission, recovery, survival, and/or clinical outcome. On the other hand, a decrease in the expression level of the OIP5 gene to the poor prognosis control level indicates a more favorable prognosis of the patient and a similarity in the expression level to the poor prognosis control level indicates less favorable, poorer prognosis for post-treatment remission, recovery, survival, and/or clinical outcome.

The expression level of the OIP5 gene in a biological sample can be considered altered when the expression level differs from the control level by more than 1.0, 1.5, 2.0, 5.0, 10.0, or more fold.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, may be used to normalize the expression levels of the OIP5 genes.

The expression level may be determined by detecting the gene transcript in the patient-derived biological sample using techniques well known in the art. The gene transcripts detected by the present method include both the transcription and translation products, such as mRNA and protein.

For instance, the transcription product of the OIP5 gene can be detected by hybridization, e.g., Northern blot hybridization analyses, that use a OIP5 gene probe to the gene transcript. The detection may be carried out on a chip or an array. The use of an array is preferable for detecting the expression level of a plurality of genes including the OIP5 gene. As another example, amplification-based detection methods, such as reverse-transcription based polymerase chain reaction (RT-PCR) which use primers specific to the OIP5 gene may be employed for the detection (see Example). The OIP5 gene-specific probe or primers may be designed and prepared using conventional techniques by referring to the whole sequence of the OIP5 gene (SEQ ID NO: 13). For example, the primers (SEQ ID NOs: 1 and 2) used in the Example may be employed for the detection by RT-PCR, but the present invention is not restricted thereto.

Specifically, a probe or primer used for the present method hybridizes under stringent, moderately stringent, or low stringent conditions to the mRNA of the OIP5 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

Alternatively, the translation product may be detected for the assessment of the present invention. For example, the quantity of the OIP5 protein may be determined. A method for determining the quantity of the protein as the translation product includes immunoassay methods that use an antibody specifically recognizing the OIP5 protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to the OIP5 protein. Methods to prepare these kinds of antibodies for the detection of proteins are well known in the art, and any method may be employed in the present invention to prepare such antibodies and equivalents thereof.

As another method to detect the expression level of the OIP5 gene based on its translation product, the intensity of staining may be observed via immunohistochemical analysis using an antibody against OIP5 protein. Namely, the observation of strong staining indicates increased presence of the OIP5 protein and at the same time high expression level of the OIP5 gene.

Furthermore, the OIP5 protein is known to have a cell proliferating activity. Therefore, the expression level of the OIP5 gene can be determined using such cell proliferating activity as an index. For example, cells which express OIP5 are prepared and cultured in the presence of a biological sample, and then by detecting the speed of proliferation, or by measuring the cell cycle or the colony forming ability the cell proliferating activity of the biological sample can be determined.

Moreover, in addition to the expression level of the OIP5 gene, the expression level of other lung and/or esophageal cancer-associated genes, for example, genes known to be differentially expressed in lung and/or esophageal cancer may also be determined to improve the accuracy of the assessment. Examples of such other lung and/or esophageal cell-associated genes include those described in WO 2004/031413 and WO 2005/090603, the contents of which are incorporated by reference herein.

Alternatively, according to the present invention, an intermediate result may also be provided in addition to other test results for assessing the prognosis of a subject. Such intermediate result may assist a doctor, nurse, or other practitioner to assess, determine, or estimate the prognosis of a subject. Additional information that may be considered, in combination with the intermediate result obtained by the present invention, to assess prognosis includes clinical symptoms and physical conditions of a subject.

The patient to be assessed for the prognosis of cancer according to the method is preferably a mammal and includes human, non-human primate, mouse, rat, dog, cat, horse, and cow.

A Kit for Diagnosing Cancer, Assessing the Prognosis of Cancer and/or Monitoring the Efficacy of a Cancer Therapy:

The present invention provides a kit for diagnosing cancer, assessing the prognosis of cancer, and/or monitoring the efficacy of a cancer therapy. Preferably, the cancer is lung and/or esophageal cancer. Specifically, the kit includes at least one reagent for detecting the expression of the OIP5 gene in a patient-derived biological sample, which reagent may be selected from the group of:

(a) a reagent for detecting mRNA of the OIP5 gene;

(b) a reagent for detecting the OIP5 protein; and

(c) a reagent for detecting the biological activity of the OIP5 protein.

Suitable reagents for detecting mRNA of the OIP5 gene include nucleic acids that specifically bind to or identify the OIP5 mRNA, such as oligonucleotides which have a complementary sequence to a part of the OIP5 mRNA. These kinds of oligonucleotides are exemplified by primers and probes that are specific to the OIP5 mRNA. These kinds of oligonucleotides may be prepared based on methods well known in the art. If needed, the reagent for detecting the OIP5 mRNA may be immobilized on a solid matrix. Moreover, more than one reagent for detecting the OIP5 mRNA may be included in the kit.

On the other hand, suitable reagents for detecting the OIP5 protein include antibodies to the OIP5 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the OIP5 protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the OIP5 protein may be included in the kit.

Furthermore, the biological activity can be determined by, for example, measuring the cell proliferating activity due to the expressed OIP5 protein in the biological sample. For example, the cell is cultured in the presence of a patient-derived biological sample, and then by detecting the speed of proliferation, or by measuring the cell cycle or the colony forming ability the cell proliferating activity of the biological sample can be determined. If needed, the reagent for detecting the OIP5 mRNA may be immobilized on a solid matrix. Moreover, more than one reagent for detecting the biological activity of the OIP5 protein may be included in the kit.

The kit may contain more than one of the aforementioned reagents. Furthermore, the kit may include a solid matrix and reagent for binding a probe against the OIP5 gene or antibody against the OIP5 protein, a medium and container for culturing cells, positive and negative control reagents, and a secondary antibody for detecting an antibody against the OIP5 protein. For example, tissue samples obtained from patient with good prognosis or poor prognosis may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be retained in a container with a label. Suitable containers include bottles, vials, and test tubes. The containers may be formed from a variety of materials, such as glass or plastic.

As an embodiment of the present invention, when the reagent is a probe against the OIP5 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of OIP5 mRNA present in the sample. The detection sites may be configured in any suitably detectable shape and are typically in the shape of a bar or dot spanning the width of a test strip.

The kit of the present invention may further include a positive control sample or OIP5 standard sample. The positive control sample of the present invention may be prepared by collecting OIP5 positive blood samples and then those OIP5 level are assayed. Alternatively, purified OIP5 protein or polynucleotide may be added to OIP5 free serum to form the positive sample or the OIP5 standard.

Screening for an Anti-Cancer Compound:

In the context of the present invention, agents to be identified through the present screening methods include any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, Ribozymes, and aptamer etc.) and natural compounds can be used in the screening methods of the present invention. The test agent of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA is confirmed it's usefulness in preparing the test agent which is a candidate for treating or preventing cancer.

Test agents useful in the screenings described herein can also be antibodies that specifically bind to OIP5 protein or partial peptides thereof that lack the biological activity of the original proteins in vivo.

Although the construction of test agent libraries is well known in the art, herein below, additional guidance in identifying test agents and construction libraries of such agents for the present screening methods are provided.

(i) Molecular Modeling:

Construction of test agent libraries is facilitated by knowledge of the molecular structure of compounds known to have the properties sought, and/or the molecular structure of OIP5. One approach to preliminary screening of test agents suitable for further evaluation utilizes computer modeling of the interaction between the test agent and its target.

Computer modeling technology allows for the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles have been published on the subject of computer modeling of drugs interactive with specific proteins, examples of which include Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents suited to the treatment and/or prophylaxis of cancer and/or the prevention of post-operative recurrence of cancer, particularly wherein the lung and/or esophageal cancer.

(ii) Combinatorial Chemical Synthesis:

Combinatorial libraries of test agents may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of Combinatorial Chemical Libraries is Well Known to Those of Skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

(iii) Other Candidates:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Aptamers are macromolecules composed of nucleic acid that bind tightly to a specific molecular target. Tuerk and Gold (Science. 249:505-510 (1990)) discloses SELEX (Systematic Evolution of Ligands by Exponential Enrichment) method for selection of aptamers. In the SELEX method, a large library of nucleic acid molecules (e.g., 10¹⁵ different molecules) can be used for screening.

Screening for an OIP5 Binding Compound:

In context of the present invention, over-expression of OIP5 was detected in lung and/or esophageal cancer, in spite of no expression in normal organs (FIGS. 1A, B, D and 2A). Accordingly, using the OIP5 genes, proteins encoded by the genes, the present invention provides a method of screening for a compound that binds to OIP5. Due to the expression of OIP5 in lung and/or esophageal cancer, a compound binds to OIP5 is expected to suppress the proliferation of cancer cells, and thus be useful for treating or preventing cancer, wherein the cancer is lung and/or esophageal. Therefore, the present invention also provides a method of screening for a compound that suppresses the proliferation of cancer cells and/or cellular invasion, and a method of screening for a compound for treating or preventing cancer using the OIP5 polypeptide, particularly wherein the cancer is lung and/or esophageal. One particular embodiment of this screening method includes the steps of:

(a) contacting a test compound with a polypeptide encoded by a polynucleotide of OIP5;

(b) detecting the binding activity between the polypeptide and the test compound; and

(c) selecting the test compound that binds to the polypeptide.

In the context of the present invention, the therapeutic effect may be correlated with the binding level of the test agent or compound and OIP5 protein(s). For example, when the test agent or compound binds to an OIP5 protein, the test agent or compound may identified or selected as a candidate agent or compound having the requisite therapeutic effect. Alternatively, when the test agent or compound does not binds to OIP5 proteins, the test agent or compound may identified as the agent or compound having no significant therapeutic effect.

The method of the present invention will be described in more detail below.

The OIP5 polypeptide to be used for screening may be a recombinant polypeptide or a protein derived from the nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to the OIP5 polypeptide using the OIP5 polypeptide, many methods well known by a person skilled in the art can be used. Such a screening can be conducted by, for example, immunoprecipitation method, specifically, in the following manner. The gene encoding the OIP5 polypeptide is expressed in host (e.g., animal) cells and so on by inserting the gene to an expression vector for foreign genes, such as pSV2neo, pcDNA I, pcDNA3.1, pCAGGS and pCD8.

The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by the OIP5 gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the OIP5 polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the OIP5 polypeptide (Experimental Medicine 13: 85-90 (1995)).

In immunoprecipitation, an immune complex is formed by adding these antibodies to cell lysate prepared using an appropriate detergent. The immune complex consists of the OIP5 polypeptide, a polypeptide including the binding ability with the polypeptide, and an antibody. Immunoprecipitation can be also conducted using antibodies against the OIP5 polypeptide, besides using antibodies against the above epitopes, which antibodies can be prepared as described above. An immune complex can be precipitated, for example by Protein A sepharose or Protein G sepharose when the antibody is a mouse IgG antibody. If the polypeptide encoded by OIP5 gene is prepared as a fusion protein with an epitope, such as GST, an immune complex can be formed in the same manner as in the use of the antibody against the OIP5 polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the OIP5 polypeptide is difficult to detect by a common staining method, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

West-Western blotting analysis (Skolnik et al., Cell 65: 83-90 (1991)) can be used as a method of screening for proteins binding to the OIP5 polypeptide using the polypeptide. In particular, a protein binding to the OIP5 polypeptide can be obtained by preparing a cDNA library from cultured cells expected to express a protein binding to the OIP5 polypeptide using a phage vector (e.g., ZAP), expressing the protein on LB-agarose, fixing the protein expressed on a filter, reacting the purified and labeled OIP5 polypeptide with the above filter, and detecting the plaques expressing proteins bound to the OIP5 polypeptide according to the label. The polypeptide of the invention may be labeled by utilizing the binding between biotin and avidin, or by utilizing an antibody that specifically binds to the OIP5, or a peptide or polypeptide (for example, GST) that is fused to the OIP5 polypeptide. Methods using radioisotope or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A compound binding to the polypeptide encoded by OIP5 gene can also be screened using affinity chromatography. For example, the polypeptide of the invention may be immobilized on a carrier of an affinity column, and a test compound, containing a protein capable of binding to the polypeptide of the invention, is applied to the column. A test compound herein may be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor such as BIAcore.

The methods of screening for molecules that bind when the immobilized OIP5 polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the OIP5 protein (including agonist and antagonist) are well known to one skilled in the art.

Screening for a Compound that Suppresses the Biological Activity of OIP5:

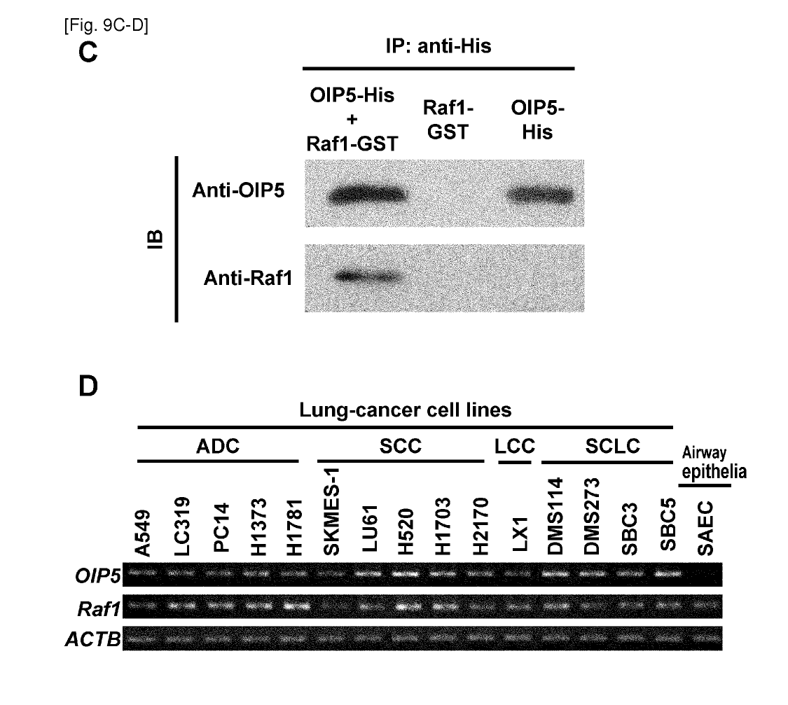

In the context of the present invention, the OIP5 protein is characterized as having the activity of promoting cell proliferation of lung and/or esophageal cancer cells (FIG. 3B) and cellular invasion activity (FIG. 9B). Using this biological activity as an index, the present invention provides a method for screening a compound that suppresses the proliferation of cancer cells and/or cellular invasion, and a method of screening for a compound for treating or preventing cancer, particularly wherein the cancer is lung and/or esophageal. Thus, the present invention provides a method of screening for a compound for treating or preventing cancer relating to OIP5 gene including the steps as follows:

(a) contacting a test compound with a polypeptide encoded by a polynucleotide of OIP5;

(b) detecting the biological activity of the polypeptide of step (a); and

(c) selecting the test compound that suppresses the biological activity of the polypeptide encoded by the polynucleotide of OIP5 as compared to the biological activity of said polypeptide detected in the absence of the test compound.

According to the present invention, the therapeutic effect of the test compound on suppressing the activity to promote cell proliferation, or a candidate compound for treating or preventing cancer relating to OIP5 (e.g., lung and/or esophageal cancers) may be evaluated. Therefore, the present invention also provides a method of screening for a candidate compound for suppressing the cell proliferation, or a candidate compound for treating or preventing cancer relating to OIP5, using the OIP5 polypeptide or fragments thereof including the steps as follows:

(a) contacting a test compound with the OIP5 polypeptide or a functional fragment thereof; and

(b) detecting the biological activity of the polypeptide or fragment of step (a), and

(c) correlating the biological activity of b) with the therapeutic effect of the test agent or compound.

In the context of present invention, the therapeutic effect may be correlated with the biological activity of an OIP5 polypeptide or a functional fragment thereof. For example, when the test agent or compound suppresses or inhibits the biological activity of an OIP5 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the biological activity of an OIP5 polypeptide or a functional fragment thereof as compared to a level detected in the absence of the test agent or compound, the test agent or compound may identified as the agent or compound having no significant therapeutic effect.

The method of the present invention will be described in more detail below.

Any polypeptides can be used for screening so long as they suppress a biological activity of an OIP5 protein. Such biological activity includes cell-proliferating activity of the OIP5 protein. For example, OIP5 protein can be used and polypeptides functionally equivalent to these proteins can also be used. Such polypeptides may be expressed endogenously or exogenously by cells.

The compound isolated by this screening is a candidate for antagonists of the polypeptide encoded by OIP5 gene. The term “antagonist” refers to molecules that inhibit the function of the polypeptide by binding thereto. This term also refers to molecules that reduce or inhibit expression of the gene encoding OIP5. Moreover, a compound isolated by this screening is a candidate for compounds which inhibit the in vivo interaction of the OIP5 polypeptide with molecules (including DNAs and proteins).

When the biological activity to be detected in the present method is cell proliferation, it can be detected, for example, by preparing cells which express the OIP5 polypeptide, culturing the cells in the presence of a test compound, and determining the speed of cell proliferation, measuring the cell cycle and such, as well as by measuring survival cells or the colony forming activity, for example, shown in FIG. 3. The compounds that reduce the speed of proliferation of the cells expressed OIP5 are selected as candidate compound for treating or preventing lung and/or esophageal cancer.

More specifically, the method includes the step of:

(a) contacting a test compound with cells expressing OIP5;

(b) measuring cell-proliferating activity; and

(c) selecting the test compound that reduces the cell-proliferating activity in the comparison with the cell-proliferating activity in the absence of the test compound.

In preferable embodiments, the method of the present invention may further include the steps of:

(d) selecting the test compound that have no effect to the cells no or little expressing OIP5.

The phrase “suppress the biological activity” as defined herein are preferably at least 10% suppression of the biological activity of OIP5 in comparison with in absence of the compound, more preferably at least 25%, 50% or 75% suppression and most preferably at 90% suppression.

Screening for a Compound Altering the Expression of OIP5:

In the present invention, a decrease in the expression of OIP5 by siRNA results in the inhibition of cancer cell proliferation (FIG. 3A) and increase in the over-expression of OIP results in the promotion of cellular invasion. Accordingly, the present invention provides a method of screening for a compound that inhibits the expression of OIP5. A compound that inhibits the expression of OIP5 is expected to suppress the proliferation of cancer cells and/or cellular invasion, and thus is useful for treating or preventing cancer relating to OIP5, particularly wherein the cancer is lung and/or esophageal. Therefore, the present invention also provides a method for screening a compound that suppresses the proliferation of cancer cells, and a method for screening a compound for treating or preventing cancer relating to OIP5, wherein the cancer is lung and/or esophageal. In the context of the present invention, such screening may include, for example, the following steps:

(a) contacting a candidate compound with a cell expressing OIP5; and

(b) selecting the candidate compound that reduces the expression level of OIP5 as compared to a control.

The method of the present invention will be described in more detail below.

Cells expressing the OIP5 include, for example, cell lines established from lung and/or esophageal cancer or cell lines transfected with OIP5 expression vectors; any of such cells can be used for the above screening of the present invention. The expression level can be estimated by methods well known to one skilled in the art, for example, RT-PCR, Northern blot assay, Western blot assay, immunostaining and flow cytometry analysis. “Reduce the expression level” as defined herein are preferably at least 10% reduction of expression level of OIP5 in comparison to the expression level in absence of the compound, more preferably at least 25%, 50% or 75% reduced level and most preferably at 95% reduced level. The compound herein includes chemical compound, double-strand nucleotide, and so on. The preparation of the double-strand nucleotide is in aforementioned description. In the method of screening, a compound that reduces the expression level of OIP5 can be selected as candidate compounds to be used for the treatment or prevention of lung and/or esophageal cancer.

Alternatively, the screening method of the present invention may include the following steps:

(a) contacting a candidate compound with a cell into which a vector, including the transcriptional regulatory region of OIP5 and a reporter gene that is expressed under the control of the transcriptional regulatory region, has been introduced;

(b) measuring the expression or activity of said reporter gene; and

(c) selecting the candidate compound that reduces the expression or activity of said reporter gene.

According to the present invention, the therapeutic effect of the test agent or compound on inhibiting the cell growth or a candidate agent or compound for treating or preventing OIP5 associating disease may be evaluated. Therefore, the present invention also provides a method for screening a candidate agent or compound that suppresses the proliferation of cancer cells, and a method for screening a candidate agent or compound for treating or preventing an OIP5 associated disease.

In the context of the present invention, such screening may include, for example, the following steps:

a) contacting a test agent or compound with a cell into which a vector, composed of the transcriptional regulatory region of the OIP5 gene and a reporter gene that is expressed under the control of the transcriptional regulatory region, has been introduced;

b) detecting the expression or activity of said reporter gene; and

c) correlating the expression level of b) with the therapeutic effect of the test agent or compound.

In the context of the present invention, the therapeutic effect may be correlated with the expression or activity of said reporter gene. For example, when the test agent or compound reduces the expression or activity of said reporter gene as compared to a level detected in the absence of the test agent or compound, the test agent or compound may identified or selected as the candidate agent or compound having the therapeutic effect. Alternatively, when the test agent or compound does not reduce the expression or activity of said reporter gene as compared to a level detected in the absence of the test agent or compound, the test agent or compound may identified as the agent or compound having no significant therapeutic effect.

Suitable reporter genes and host cells are well known in the art. Illustrative reporter genes include, but are not limited to, luciferase, green florescence protein (GFP), Discosoma sp. Red Fluorescent Protein (DsRed), Chrolamphenicol Acetyltransferase (CAT), lacZ and beta-glucuronidase (GUS), and host cell is COS7, HEK293, HeLa and so on. The reporter construct required for the screening can be prepared by connecting reporter gene sequence to the transcriptional regulatory region of OIP5. The transcriptional regulatory region of OIP5 herein includes the region from transcriptional start site to at least 500 bp upstream, preferably 1000 bp, more preferably 5000 or 10000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press).

The vector containing the said reporter construct is infected to host cells and the expression or activity of the reporter gene is detected by method well known in the art (e.g., using luminometer, absorption spectrometer, flow cytometer and so on). “reduces the expression or activity” as defined herein are preferably at least 10% reduction of the expression or activity of the reporter gene in comparison with in absence of the compound, more preferably at least 25%, 50% or 75% reduction and most preferably at 95% reduction.

Screening Using the Binding of OIP5 and Raf1 as an Index:

In the present invention, the direct interaction of OIP5 with Raf1 protein was shown by pull-down assay (FIG. 9C). Pull-down of OIP5 protein was carried out using anti-His antibody and incubated mixture of His-tagged OIP5 and GST-fused recombinant Rafa proteins. OIP5-binding Raf1 protein was detected by subsequent western blotting using polyclonal antibody to Raf1 (FIG. 9C). Accordingly, the present invention provides a method of screening for a compound that inhibits the binding between OIP5 and Raf1.

Compounds that inhibits the binding between OIP5 protein and Raf1 protein can be screened by detecting a binding level between OIP5 protein and Raf1 protein as an index. Therefore, the present invention provides a method for screening a compound for inhibiting the binding between OIP5 protein and Raf1 protein using a binding level between OIP5 protein and Raf1 protein as an index. Compounds that inhibit binding between OIP5 protein and Raf1 protein are expected to be suppress cancer cell proliferation and/or cellular invasion through destabilization of OIP5 protein. Accordingly, the present invention also provides a method for screening a candidate compound for inhibiting or reducing a growth of cancer cells expressing OIP5 gene, e.g., lung cancer cell and/or esophageal cancer cell, and therefore, a candidate compound for treating or preventing cancers, e.g. lung cancer and/or esophageal cancer. Further, compounds obtained by the present screening method may be also useful for inhibiting cellular invasion.

Of particular interest to the present invention are the following methods of [1] to [5]:

[1] A method of screening for a compound that interrupts a binding between a OIP5 polypeptide and a Raf1 polypeptide, said method including the steps of:

(a) contacting a OIP5 polypeptide or functional equivalent thereof with a Raf1 polypeptide or functional equivalent thereof in the presence of a test compound;

(b) detecting a binding level between the polypeptides;

(c) comparing the binding level detected in the step (b) with those detected in the absence of the test compound; and

(d) selecting the test compound that reduce the binding level.

[2] A method of screening for an agent or compound useful in treating or preventing cancers, or inhibiting cancer cell growth and/or cellular invasion, said method including the steps of:

(a) contacting a OIP5 polypeptide or functional equivalent thereof with a Raf1 polypeptide or functional equivalent thereof in the presence of a test compound;

(b) detecting a binding level between the polypeptides;

(c) comparing the binding level detected in the step (b) with those detected in the absence of the test compound; and

(d) selecting the test compound that reduce the binding level.

[3] The method of [1] or [2], wherein the functional equivalent of OIP5 including the Raf1-binding domain.

[4] The method of [1] or [2], wherein the functional equivalent of Raf1 including the OIP5-binding domain.

[5] The method of [1], wherein the cancer is selected from the group consisting of lung cancers and esophageal cancer.

In the context of the present invention, functional equivalents of an OIP5 and Raf1 polypeptide are polypeptides that have a biological activity equivalent to a OIP5 polypeptide (SEQ ID NO: 14), Raf1 (SEQ ID NO: 18) polypeptide, respectively. Particularly, the functional equivalent of OIP5 is a poly peptide fragment containing the binding domain to Raf1, such as amino acid sequence of SEQ ID NO: 14. Similarly, the functional equivalent of Raf1 is a polypeptide fragment of SEQ ID NO: 17 including the OIP5-binding domain.

As a method of screening for compounds that inhibits, the binding of OIP5 to Raf1, many methods well known by one skilled in the art can be used.

A polypeptide to be used for screening can be a recombinant polypeptide or a protein derived from natural sources, or a partial peptide thereof. Any test compound aforementioned can be used for screening.

As a method of detecting the binding between an OIP5 protein and Raf1 protein, any methods well known by a person skilled in the art can be used. Such a detection can be conducted using, for example, an immunoprecipitation, West-Western blotting analysis (Skolnik et al., Cell 65: 83-90 (1991)), a two-hybrid system utilizing cells (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”), affinity chromatography and a biosensor using the surface plasmon resonance phenomenon.

In some embodiments, the present screening method may be carried out in a cell-based assay using cells expressing both of a OIP5 protein and a Raf1 protein. Cells expressing OIP5 protein and Raf1 protein include, for example, cell lines established from cancer, e.g. lung cancer and/or esophageal cancer. Alternatively the cells may be prepared by transforming cells with nucleotides encoding OIP5 and Raf1 protein. Such transformation may be carried out using an expression vector encoding both OIP5 and Raf1, or expression vectors encoding either OIP5 or Raf1. The present screening can be conducted by incubating such cells in the presence of a test compound. The binding of OIP5 protein to Raf1 protein can be detected by immunoprecipitation assay using an anti-OIP5 antibody or anti-Raf1 antibody.

According to the present invention, the therapeutic effect of a candidate agent or compound on inhibiting the cell growth or a candidate agent or compound for treating or preventing cancer relating to OIP5 (e.g., lung and esophageal cancers) may be evaluated. Therefore, the present invention also provides a method of screening for a candidate agent or compound for suppressing the cell proliferation, or a candidate agent or compound for treating or preventing cancer (e.g., lung and esophageal cancers), using a OIP5 polypeptide or functional equivalent thereof including the steps of:

(a) contacting a OIP5 polypeptide or functional equivalent thereof with a Raf1 polypeptide or functional equivalent thereof in the presence of a test agent or compound;

(b) detecting a binding level between the polypeptides;

(c) comparing the binding level detected in the step (b) with those detected in the absence of the test agent or compound; and

(d) correlating the binding level of (c) with the therapeutic effect of the test agent or compound;

In the present invention, the therapeutic effect may be correlated with the binding level between a OIP5 polypeptide and a Raf1 polypeptide. For example, when the test agent or compound suppresses the binding level between the polypeptides as compared to a level detected in the absence of the test agent or compound, the test agent or compound may identified or selected as the candidate agent or compound having the therapeutic effect. Alternatively, when the test agent or compound does not suppress or inhibit the binding level between the polypeptides as compared to a level detected in the absence of the test agent or compound, the test agent or compound may identified as the agent or compound having no significant therapeutic effect.

Screening for a Compound that Suppresses the Phosphorylation of OIP5:

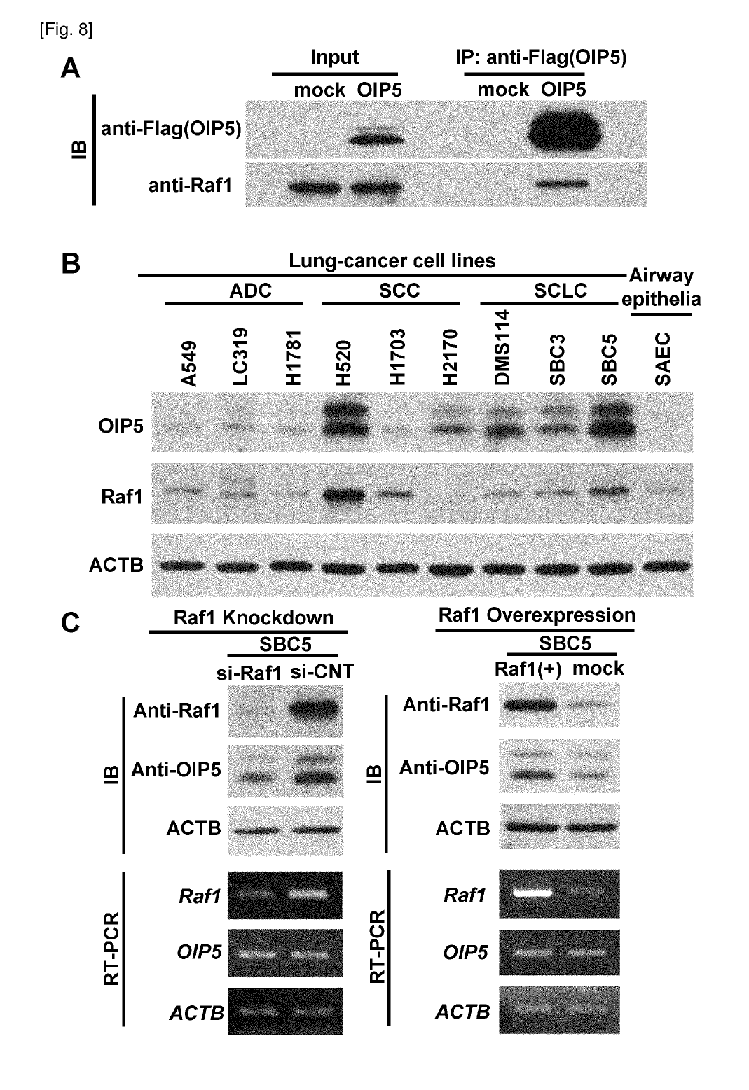

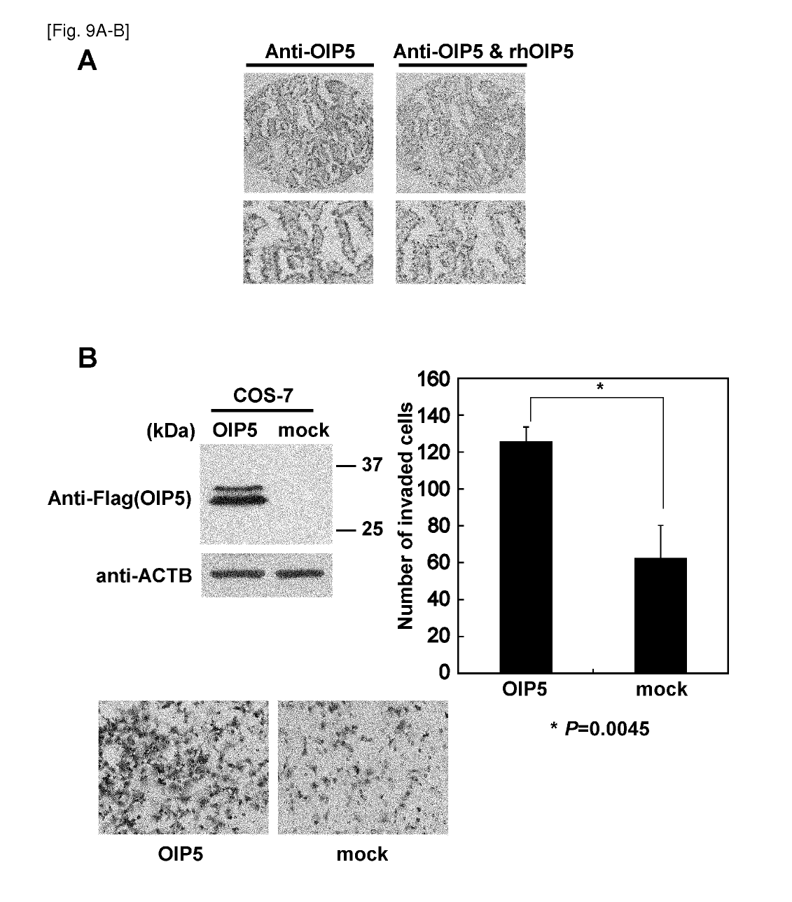

In the present invention, the phosphorylation of an OIP5 protein by a Raf1 protein is demonstrated to contributes the stabilization of the OIP5 polypeptide (FIG. 8C), and therefore, it may have a crucial role in cancer cell growth. Accordingly, compounds that inhibit the phosphorylation of an OIP5 protein by a Raf1 protein are expected to be useful for inhibiting cancer cell growth and/or cellular invasion, and therefore, may be candidate compounds for treating or preventing cancer relating to OIP5 over-expression (e.g., lung cancer or esophageal cancer).

Therefore, the present invention also provide a method of screening a candidate compound for suppressing cell proliferation and/or cellular invasion, or a candidate compound for treating or preventing cancer relating to OIP5 over-expression using the phosphorylation level of OIP5 as an index. In the context of the present invention, such screening may include, for example, the following steps:

(a) contacting an OIP5 polypeptide or a functional equivalent thereof with a Raf1 polypeptide or a functional equivalent thereof in the presence of a test compound under a suitable condition for the phosphorylation of the OIP5 polypeptide;

(b) detecting the phosphorylation level of the OIP5 polypeptide;

(c) comparing the phosphorylation level in the step (b) with those detected in the absence of the test compound; and

(d) selecting the test compound that reduces the phosphorylation level of the OIP5 polypeptide.

According to the present invention, the therapeutic effect of the test agent or compound on inhibiting the cell growth or a candidate agent or compound for treating or preventing OIP5 associating disease, e.g., lung cancer and esophageal cancer, may be evaluated. Therefore, the present invention also provides a method for screening a candidate agent or compound that suppresses the proliferation of breast cancer cells, and a method for screening a candidate agent or compound for treating or preventing breast cancer.

More specifically, the method includes the steps of:

(a) contacting an OIP5 protein with a Raf1 protein in the presence of an test agent or compound;

(b) detecting the phosphorylation level of the OIP5 protein;

(c) comparing the phosphorylation level of the OIP5 protein with that detected in the absence of the test agent or compound; and

(d) correlating the phosphorylation level of c) with the therapeutic effect of the test agent or compound.

In the present invention, the therapeutic effect may be correlated with the phosphorylation level of the OIP5 protein. For example, when the test agent or compound reduces the phosphorylation level of the OIP5 protein as compared to a level detected in the absence of the test agent or compound, the test agent or compound may identified or selected as the candidate agent or compound having the therapeutic effect. Alternatively, when the test agent or compound does not reduce the phosphorylation level of OIP5 protein as compared to a level detected in the absence of the test agent or compound, the test agent or compound may identified as the agent or compound having no significant therapeutic effect.

In the context of the present invention, a functional equivalent of a OIP5 or Raf1 polypeptide is a polypeptide that has a biological activity equivalent to a OIP5 polypeptide (SEQ ID NO: 14) or Raf1 polypeptide (SEQ ID NO: 18), respectively. As used herein, a phrase “functional equivalent” is the same meaning as described in the item “Genes or Proteins”.

As a method of screening for compounds that inhibit the phosphorylation of an OIP5 polypeptide by a Raf1 polypeptide, any method known in the art can be used. In the context of the present invention, the conditions suitable for the phosphorylation of an OIP5 polypeptide may be provided with an incubation of an isolated OIP5 polypeptide and an isolated Raf1 polypeptide in the presence of a phosphate donor, e.g., ATP. The conditions suitable for the OIP5 phosphorylation by Raf1 also include culturing cells expressing the both of an OIP5 polypeptide and a Raf1 polypeptide. For example, such a cell may be a cell that endogenously expresses an OIP5 and Raf1 such as a cancer cell (e.g., a lung cancer cell, an esophageal cancer cell), or a transformant cell harboring expression vectors containing polynucleotides that encode an OIP5 polypeptide and/or a Raf1 polypeptide.

After the incubation of the isolated polypeptides or cells expressing the polypeptides in the presence or a test compound, the phosphorylation level of the OIP5 polypeptide can be detected with a reagent, such as an antibody recognizing phosphorylated OIP5. For instance, immunoassay or Western-blotting assay may be applied to the detection of the phosphorylation state of OIP5 polypeptide.

Prior to the detection of phosphorylated OIP5, the OIP5 polypeptide may be separated from other elements. For instance, gel electrophoresis may be used for the separation of the OIP5 polypeptide from remaining components. Alternatively, an OIP5 polypeptide may be captured by a carrier having an anti-LGN/GPSM2 antibody.

Alternatively, the phosphorylation level of the OIP5 polypeptide may be detected by incubating an isolated OIP5 polypeptide and Raf1 polypeptide, or cells expressing these polypeptides with a labeled phosphate donor, and then tracing the label. For example, when radio-labeled ATP (e.g., 32P-ATP) is used as a phosphate donor, radio activity of the separated OIP5 polypeptide correlates with the phosphorylation level of OIP5 polypeptide.

In the context of the present invention, candidate compounds that have the potential to treat or prevent cancers can be identified. The therapeutic potential of these candidate compounds may be evaluated by second and/or further screening to identify therapeutic agent for cancers. For example, when a compound binding to OIP5 protein inhibits described above activities of the cancer, it may be concluded that such compound has the OIP5 specific therapeutic effect.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### EXAMPLES

The invention will be further described in the following examples, which do not limit the scope of the invention described in the claims.

Materials and Methods

Cell Lines and Tissue Samples.

The 15 human lung-cancer cell lines used in this study included five adeno-carcinomas (ADCs) (A549, LC319, PC-14, NCI-H1373, and NCI-H1781), five squamous-cell carcinomas (SCCs) (SK-MES-1, LU61, NCI-H520, NCI-H1703, and NCI-H2170), one large-cell carcinoma (LCC) (LX1), and four small-cell lung cancers (SCLCs) (DMS114, DMS273, SBC-3, and SBC-5). The human esophageal carcinoma cell lines used in this study were as follows; 9 SCC cell lines (TE1, TE2, TE3, TE4, TE5, TE6, TE8, TE9, and TE10) and one ADC cell line (TE7) (Nishihira T, et al., J Cancer Res Clin Oncol 1993; 119:441-9). All cells were grown in monolayer in appropriate media supplemented with 10% fetal calf serum (FCS) and were maintained at 37 degrees C. in humidified air with 5% CO₂. Human small airway epithelial cells (SAEC) used as a normal control were grown in optimized medium (SAGM) from Cambrex Bio Science Inc. Primary lung cancer and ESCC samples had been obtained earlier. This study and the use of all clinical materials mentioned were approved by individual institutional Ethical Committees. Clinical stage was judged according to the UICC TNM classification (Sobin L and Wittekind Ch. 6th ed. New York: Wiley-Liss; 2002). Formalin-fixed primary lung tumors and adjacent normal lung tissue samples used for immunostaining on tissue microarrays had been obtained from 279 patients (161 ADCs, 96 SCCs, 18 LCCs, 4 ASCs; 96 female and 183 male patients; median age of 63.3 with a range of 26-84 years) undergoing curative surgery at Hokkaido University (Sapporo, Japan). A total of 280 formalin-fixed primary ESCCs (27 female and 253 male patients; median age of 61.5 with a range of 38-82 years) and adjacent normal esophageal tissue samples had also been obtained from patients undergoing curative surgery at Keiyukai Sapporo Hospital (Sapporo, Japan). Further, a total of 336 NSCLCs (stage I-IIIA; 201 ADCs, 101 SCCs, 23 LCCs, 11 ASCs; 103 female and 233 male patients; median age of 66.0 with a range of 29-85 years) and normal lung tissue samples for immunostaining on tissue microarray were also obtained from Saitama Cancer Center (Saitama, Japan). These patients received resection of their primary cancers, and among them only patients with positive lymph node metastasis were treated with platinum-based adjuvant chemotherapies after their surgery. Formalin-fixed primary 221 ESCCs (stage I-IVA; 21 female and 200 male patients; median age of 62 with a range of 44-79 years) and adjacent normal esophageal tissue samples had been obtained from patients undergoing curative surgery at Keiyukai Sapporo Hospital (Sapporo, Japan). 76 ESCCs (stage I-IVB; 13 female and 63 male patients; median age of 63 with a range of 38-84 years) and adjacent normal esophageal tissue samples had also been obtained from patients undergoing curative surgery Hokkaido University and its affiliated hospitals (Sapporo, Japan). This study and the use of all clinical materials were approved by individual institutional ethical committees.

Semiquantitative RT-PCR.

Total RNA was extracted from cultured cells and clinical tissues using Trizol reagent (Life Technologies, Inc., Gaithersburg, Md.) according to the manufacturer's protocol. Extracted RNAs and normal human tissue poly(A) RNAs were treated with DNase I (Nippon Gene, Tokyo, Japan) and reversely-transcribed using oligo (dT) primer and SuperScript II reverse transcriptase (Invitrogen, Carlsbad, Calif.). Semiquantitative RT-PCR experiments were carried out with the following OIP5-specific primers or with ACTB-specific primers as an internal control:

| | |
|---|---|
| OIP5: 5′-CTTCAAGAATGGAGGGGAAA-3′, | (SEQ ID NO: 1) |
| and | |
| 5′-GTATTCATAACAACTGCTCCATGC-3′; | (SEQ ID NO: 2) |
| ACTB: 5′-GAGGTGATAGCATTGCTTTCG-3′ | (SEQ ID NO: 3) |
| and | |
| 5′-CAAGTCAGTGTACAGGTAAGC-3′. | (SEQ ID NO: 4) |

PCR reactions were optimized for the number of cycles to ensure product intensity within the logarithmic phase of amplification.

Northern-Blot Analysis.

Human multiple-tissue blots (BD Biosciences Clontech, Palo Alto, Calif.) were hybridized with a ³²P-labeled PCR product of OIP5. The cDNA probes of OIP5 were prepared by RT-PCR using the following primers:

| | |
|---|---|
| 5′-CCAGTGACAAAATGGTGTGC-3′, | (SEQ ID NO: 5) |
| and | |
| 5′-GTATTCATAACAACTGCTCCATGC-3′. | (SEQ ID NO: 6) |

Pre-hybridization, hybridization, and washing were performed according to the supplier's recommendations. The blots were autoradiographed at −80 degrees C. for 1 week with intensifying screens.

Western-Blotting.

Tumor tissues or cells were lysed in lysis buffer; 50 mM Tris-HCl (pH 8.0), 150 mM NaCl, 0.5% NP40, 0.5% sodium deoxycholate, and Protease Inhibitor Cocktail Set III (Calbiochem). An enhanced chemiluminescence Western blotting analysis system (GE Healthcare Biosciences) was used, as previously described (Kato T, et al., Cancer Res 2005; 65:5638-46). A commercially available rabbit polyclonal antibody to human OIP5 (Catalog No. 12142-1-AP, Proteintech group. Inc.) was confirmed to be specific to endogenous OIP5 protein by western-blot analysis using lysates of lung cancer and esophageal cancer cell lines as well as normal airway epithelial cells.

Immunocytochemistry.

Cultured cells were fixed with 4% paraformaldehyde, and permeabilized with 0.1% Triton X-100 in PBS for 3 minutes at room temperature. Cells were covered by CASBLOCK (ZYMED) for 10 minutes at room temperature to block nonspecific binding. Cells were then incubated for 60 minutes at room temperature with primary antibodies diluted in PBS containing 1% BSA. After being washed with PBS, the immunocomplexes were stained with a goat anti-rabbit secondary antibody conjugated to Alexa 488 (Invitrogen). Each specimen was mounted with Vectashield (Vector Laboratories, Inc, Burlingame, Calif.) containing 4′,6′-diamidine-2′-phenylindolendihydrochrolide (DAPI) and visualized with Spectral Confocal Scanning Systems (TSC SP2 AOBS: Leica Microsystems, Wetzlar, Germany).

Immunohistochemistry and Tissue-Microarray Analysis.

Tumor tissue microarrays were constructed as published previously, using formalin-fixed NSCLCs (Chin S F, et al., Mol Pathol 2003; 56:275-9, Callagy G, et al., Diagn Mol Pathol 2003; 12:27-34, Callagy G, et al. J Pathol 2005; 205:388-96). Tissue areas for sampling were selected based on visual alignment with the corresponding H&E stained sections on slides. Three, four, or five tissue cores (diameter, 0.6 mm; height, 3-4 mm) taken from donor tumor blocks were placed into recipient paraffin blocks using a tissue microarrayer (Beecher Instruments, Sun Prairie, Wis.). A core of normal tissue was punched from each case. Five-micrometer sections of the resulting microarray block were used for immunohistochemical analysis. Positivity for OIP5 was assessed semiquantitatively by three independent investigators without prior knowledge of the clinicopathologic data, each of whom recorded staining positive or negative. Lung cancers were decided as positive only if all reviewers defined them as such.

To investigate the significance of OIP5 overexpression in clinical NSCLCs, tissue sections were stained using ENVISION+kit/horseradish peroxidase (HRP; DakoCytomation, Glostrup, Denmark). OIP5 antibody (Proteintech group. Inc.) was added after blocking of endogenous peroxidase and proteins, and each section was incubated with HRP-labeled anti-rabbit IgG as the secondary antibody. Substrate-chromogen was added, and the specimens were counterstained with hematoxylin.

Statistical Analysis.

Contingency tables were used to analyze the relationship between OIP5 expression and clinicopathologic variables in NSCLC patients. Tumor-specific survival curves were calculated from the date of surgery to the time of death related to NSCLC, or to the last follow-up observation. Kaplan-Meier curves were calculated for each relevant variable and for OIP5 expression; differences in survival times among patient subgroups were analyzed using the log-rank test. Univariate and multivariate analyses were done with the Cox proportional hazard regression model to determine associations between clinicopathologic variables and cancer-related mortality. First, associations between death and possible prognostic factors including age, gender, histological type, pT-classification, and pN-classification, were analyzed, taking into consideration one factor at a time. Second, multivariate Cox analysis was applied on backward (stepwise) procedures that always forced OIP5 expression into the model, along with any and all variables that satisfied an entry level of a P value less than 0.05. As the model continued to add factors, independent factors did not exceed an exit level of P<0.05.

RNA Interference Assay.

Small interfering RNA (siRNA) duplexes (Dharmacon, Inc., Lafayette, Colo.) (100 nM) were transfected into a NSCLC cell line A549, using 30 micro 1 of Lipofectamine 2000 (Invitrogen, Carlsbad, Calif.) following the manufacturer's protocol. The transfected cells were cultured for 7 days, the number of colonies was counted by Giemsa staining; and viability of cells was evaluated by 3-(4,5-dimethylthiazol-2-yl)-2,5-diphenyltetrazolium bromide (MTT) assay (cell counting kit-8 solution; Dojindo Laboratories, Kumamoto, Japan). To confirm suppression of OIP5 mRNA expression, semiquantitative RT-PCR experiments were carried out with synthesized primers specific to OIP5 described above. The target sequences of the synthetic oligonucleotides for RNA interference were as follows:

control 1 (Luciferase/LUC: Photinus pyralis luciferase gene):

| | |
|---|---|
| 5′-CGUACGCGGAAUACUUCGA-3′; | (SEQ ID NO: 7) |

control 2 (On-Target plus/CNT; Dharmacon Inc.):

(SEQ ID NO: 8)
5′-UGGUUUACAUGUCGACUAA-3′;
(SEQ ID NO: 9)
siRNA-OIP5-1: 5′-CGGCAUCGCUCACGUUGUGUU-3′;
(SEQ ID NO: 10)
siRNA-OIP5-2: 5′-GUGACAAAAUGGUGUGCUAUU-3′;
(SEQ ID NO: 15)
siRNA-Raf1-1: 5′-GCAAAGAACAUCAUCCAUA-3′;
and
(SEQ ID NO: 16)
siRNA-Raf1-2: 5′-GACAUGAAAUCCAACAAUA-3′.

Cell Growth Assay.

COS-7 cells were plated at densities of 1×10⁶ cells/100 mm dish, transfected with plasmids designed to express OIP5 (pcAGGSn3FC-OIP5-Flag) or mock plasmids. Cells were selected in medium containing 0.4 mg/mL of geneticin (Invitrogen) for 7 days, and cell numbers were assessed by MTT assay (cell counting kit-8 solution; Dojindo Laboratories).

Matrigel Invasion Assay.

COS-7 cells transfected either with p3XFLAG-tagged plasmids expressing OIP5 (pcAGGSn3FC-OIP5-Flag) or with mock plasmids were grown to near confluence in DMEM containing 10% FCS. The cells were harvested by trypsinization, washed in DMEM without addition of serum or proteinase inhibitor, and suspended in DMEM at concentration of 1×10⁵ cells/ml. Before preparing the cell suspension, the dried layer of Matrigel matrix (Becton Dickinson Labware) was rehydrated with DMEM for 2 hours at room temperature. DMEM (0.75 ml) containing 10% FCS was added to each lower chamber in 24-well Matrigel invasion chambers, and 0.5 ml (5×10⁴ cells) of cell suspension was added to each insert of the upper chamber. The plates of inserts were incubated for 24 hours at 37 degrees C. After incubation the chambers were processed; cells invading through the Matrigel were fixed and stained by Giemsa as directed by the supplier (Becton Dickinson Labware).

Results

OIP5 expression in lung and esophageal cancers and normal tissues. To identify target molecules for the development of novel therapeutic agents and/or biomarkers for lung and esophageal cancers, genome-wide expression profile analysis of lung carcinoma and ESCC was performed using a cDNA microarray (Daigo Y and Nakamura Y, Gen Thorac Cardiovasc Surg 2008; 56:43-53, Kikuchi T, et al., Oncogene 2003; 22:2192-205, Kakiuchi S, et al., Mol Cancer Res 2003; 1:485-99, Kakiuchi S, et al., Hum Mol Genet 2004; 13:3029-43, Kikuchi T, et al., Int J Oncol 2006; 28:799-805, Taniwaki M, et al., Int J Oncol 2006; 29:567-75, and Yamabuki T, et al., Int J Oncol 2006; 28:1375-84). Among 27,648 genes screened, elevated expression (3-fold or higher) of OIP5 transcript was identified in the great majority of the lung and esophageal cancer samples examined. Its over-expression was confirmed by means of semi-quantitative RT-PCR experiments in 9 of 15 lung cancer tissues, in 15 of 15 lung-cancer cell lines, in 6 of 10 ESCC tissues, and in 9 of 10 ESCC cell lines (FIGS. 1A and 1B).

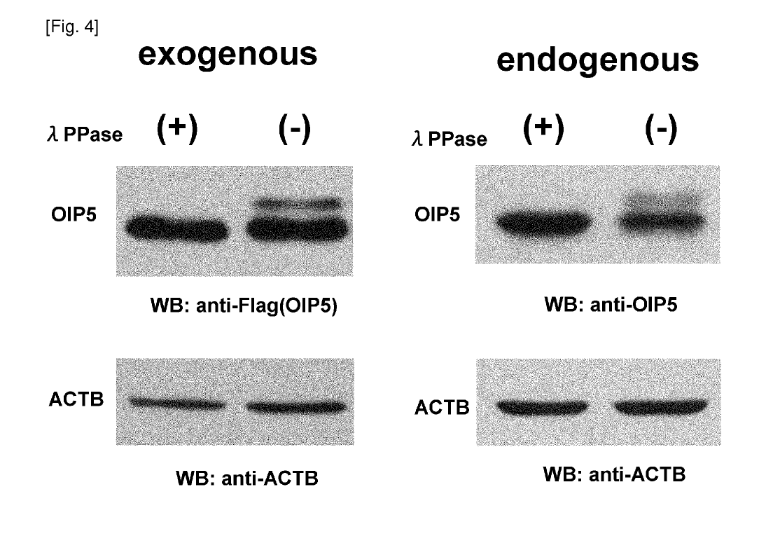

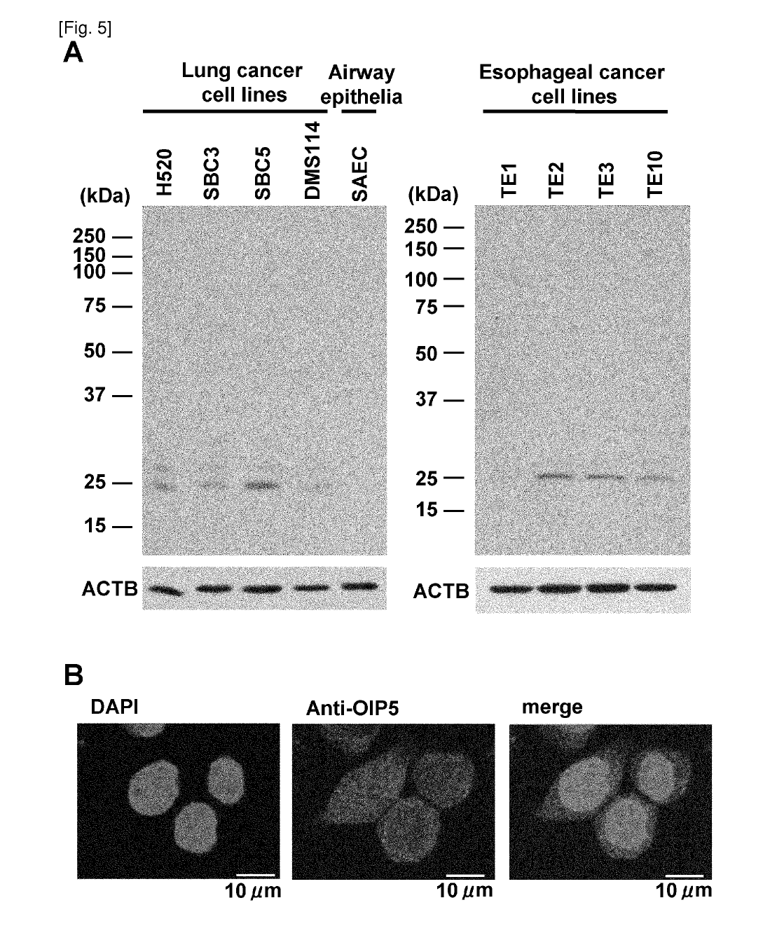

A high level of OIP5 expression in lung and esophageal cancer cell lines was further confirmed by Western blot analyses using anti-OIP5 antibody (FIG. 5A). OIP5 protein was detected as double bands by western blotting, indicating a possible modification of the OIP5 protein. Therefore, extracts from SBC-5 cells that overexpressed endogenous OIP5 and COS-7 cells transfected with OIP5 expressing plasmids (pcAGGSn3FC-OIP5-Flag) were incubated in the presence or absence of protein phosphatase (New England Biolabs), and analyzed the molecular size of OIP5 protein by western-blot analysis. The measured weight of the majority of both endogenous and exogenous OIP5 protein in the extracts treated with phosphatase was smaller than that in the untreated cells. The data indicated that OIP5 was possibly phosphorylated in cells (FIG. 4). Immunofluorescence analysis was done to examine the subcellular localization of endogenous OIP5 in a lung cancer cell line SBC-5 and found that OIP5 was located in the nucleus and cytoplasm (FIG. 1C, FIG. 5B). Northern-blot analysis using OIP5 cDNA as a probe identified a strong signal corresponding to a 1.5-kb transcript only in the testis among 16 tissues (FIG. 1D) or 23 tissues (FIG. 6A) examined. Furthermore, OIP5 protein expressions in six normal tissues (liver heart, kidney, lung, esophagus and testis) were compared with those in lung and esophageal cancers using anti-OIP5 polyclonal antibodies by immunohistochemistry. OIP5 was detected abundantly in nucleus and cytoplasm of testicular cells and lung and esophageal cancer cells; however, its expression was hardly detectable in the remaining five normal tissues (FIG. 6B). The positive signal by anti-OIP5 antibody obtained in lung cancer tissues was diminished by preincubation of the antibody with recombinant human OIP5, indicating its high specificity to OIP5 protein (FIG. 9A).

Association of OIP5 Expression with Poor Prognosis for NSCLC Patients and ESCC.

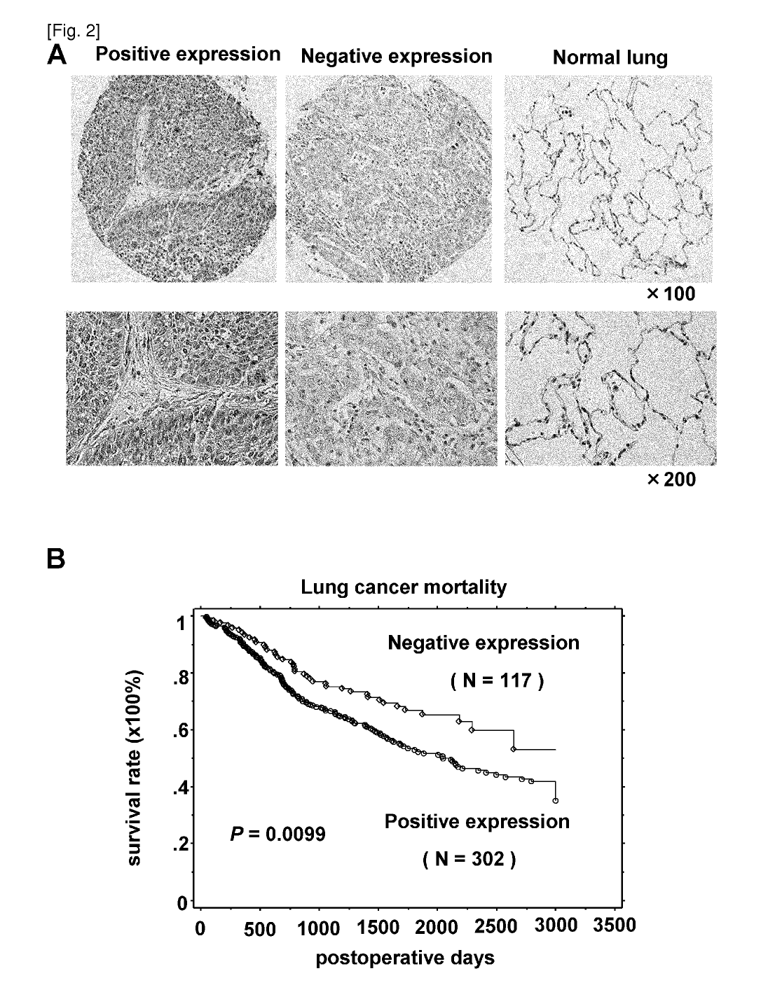

Correlations of the OIP5 expression in surgically resected NSCLCs were then examined with various clinicopathologic variables. To verify the clinicopathological significance of OIP5, the expression of OIP5 protein was additionally examined by means of tissue microarrays containing lung-cancer tissues from 419 patients who underwent curative surgical resection. Positive staining was found in 163 of 262 ADC tumors (62.2%) and 139 of 157 non-ADC tumors (88.5%) (FIG. 2A). A correlation of OIP5 expression (positive versus negative) with various clinicopathological parameters was then examined and its significant correlation with histology (higher in nonadenocarcinomas; P<0.0001 by Chi-square test; Table 1) was found. The Kaplan-Meier method indicated significant association between OIP5 status (positive versus negative) in NSCLCs and tumor-specific survival rate (shorter survival periods in OIP5-positive cases; P=0.0099 by the log-rank test; FIG. 2B). By univariate analysis, histology (adenocarcinomas versus nonadenocarcinomas), tumor size (pT1 versus pT2-4), lymph node metastasis (pN0 versus pN1-3), age (<65 years versus z65 years), gender (female versus male), and OIP5 positivity (negative versus positive) were all significantly related to poor tumor-specific survival of NSCLC patients (Table 2). Furthermore, multivariate analysis using the Cox proportional hazard model indicated that pT stage, pN stage, age, and positive OIP5 staining were independent prognostic factors for NSCLC (Table 2).

TABLE 1
Association between OIP5-positivity in NSCLC tissues and patients'
characteristics (n = 419)

| | | OIP5 | OIP5 | | P-value |
|---|---|---|---|---|---|
| | Total | positive | negative | | positive vs |
| | n = 419 | n = 302 | n = 117 | χ² | negative |
| Age (years) | | | | | |
| <65 | 207 | 153 | 54 | 0.686 | 0.686 |
| >=65 | 212 | 149 | 63 | | |
| Gender | | | | | |
| Female | 129 | 86 | 43 | 2.71 | 0.0997 |
| Male | 290 | 216 | 74 | | |
| Histological type | | | | | |
| ADC | 262 | 163 | 99 | 33.794 | <0.0001* |
| non-ADC | 157 | 139 | 18 | | |
| pT factor | | | | | |
| T1 | 141 | 96 | 45 | 1.682 | 0.1946 |
| T2 + T3 + T4 | 278 | 206 | 72 | | |
| pN factor | | | | | |
| N0 | 259 | 182 | 77 | 1.099 | 0.2944 |
| N1 + N2 | 160 | 120 | 40 | | |

ADC, adenocarcinoma
non-ADC, squamous-cell carcinoma plus large-cell carcinoma and adenosquamous-cell carcinoma
P < 0.05 (χ² test)

TABLE 2
Cox's proportional hazards model analysis of prognostic factors in
patients with NSCLCs

| | Hazards | | Unfavorable/ | |
|---|---|---|---|---|
| Variables | ratio | 95% CI | Favorable | P-value |
| Univariate | | | | |
| analysis | | | | |
| OIP5 | 1.567 | 1.110-2.212 | Positive/Negative | 0.0107* |
| Age (years) | 1.522 | 1.146-2.021 | >=65/65> | 0.0037* |
| Gender | 1.618 | 1.172-2.235 | Male/Female | 0.0035* |
| Histological type | 1.424 | 1.076-1.885 | non-ADC/ADC | 0.0135* |
| pT factor | 2.468 | 1.744-3.494 | T2 + T3 + T4/T1 | <0.0001* |
| pN factor | 2.342 | 1.771-3.099 | N1 + N2/N0 | <0.0001* |
| Multivariate | | | | |
| analysis | | | | |
| OIP5 | 1.56 | 1.083-2.247 | Positive/Negative | 0.0169* |
| Age (years) | 1.811 | 1.352-2.426 | >=65/65> | <0.0001* |
| Gender | 1.382 | 0.965-1.980 | Male/Female | 0.0774 |
| Histological type | 0.881 | 0.633-1.224 | non-ADC/ADC | 0.4494 |
| pT factor | 1.939 | 1.353-2.780 | T2 + T3 + T4/T1 | 0.0003* |
| pN factor | 2.316 | 1.728-3.104 | N1 + N2/N0 | <0.0001* |

ADC, adenocarcinoma
non-ADC, squamous-cell carcinoma plus large-cell carcinoma and adenosquamous-cell carcinoma
P < 0.05

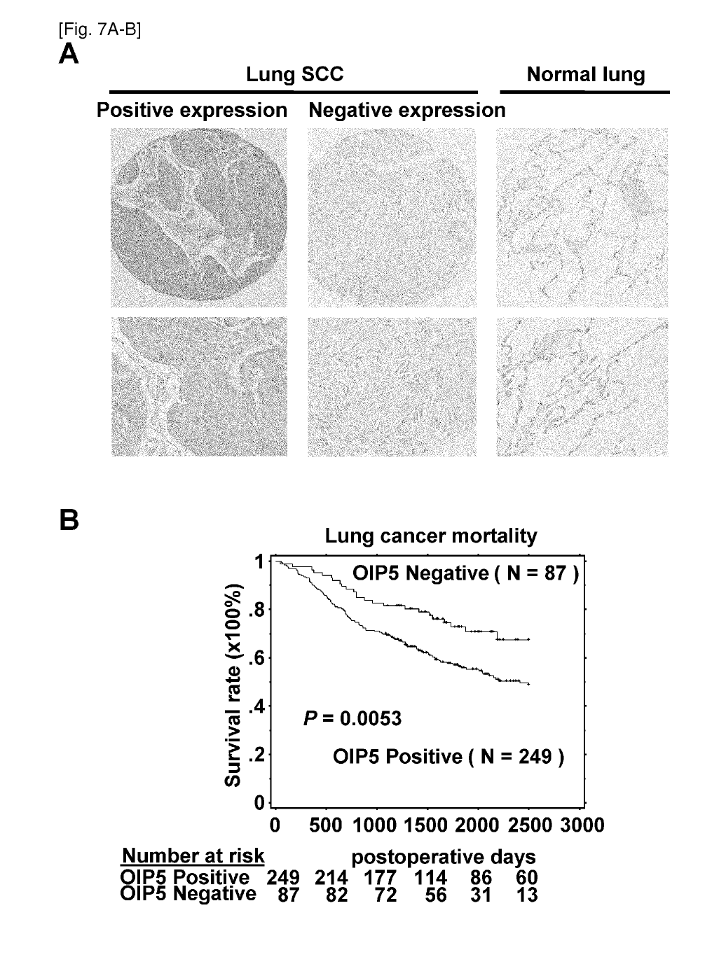

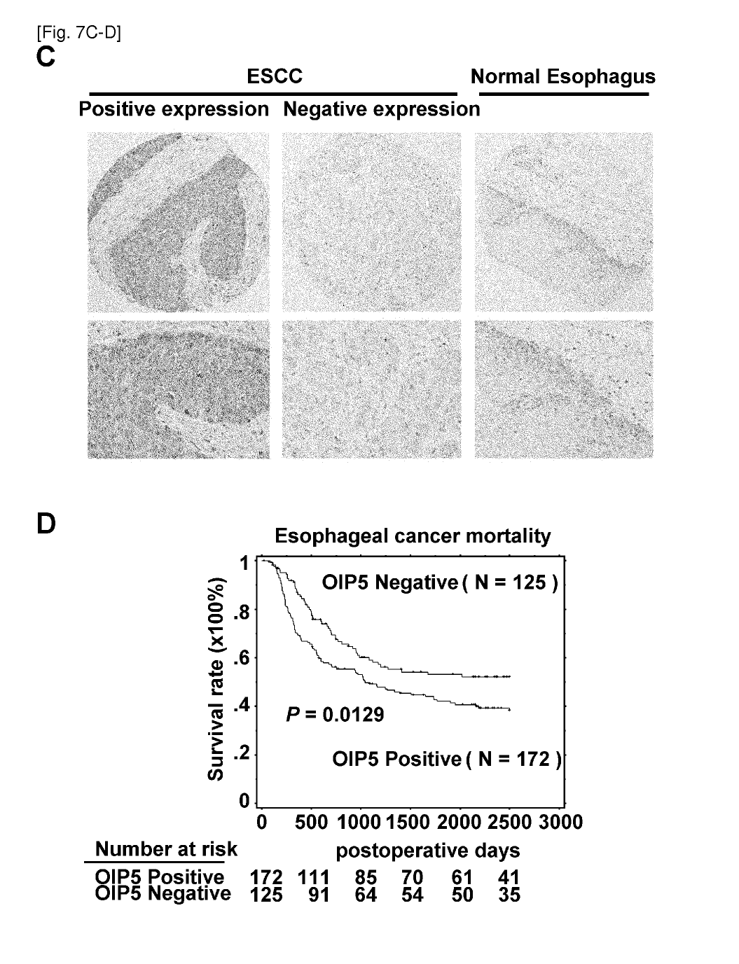

To further verify the clinicopathological significance of OIP5, additionally the expression of OIP5 protein was examined by means of tissue microarrays containing lung-cancer tissues from 336 NSCLC and 297 ESCC patients who underwent surgical resection. Positive staining were found in 131 of 201 ADC tumors (65.2%), 118 of 135 non-ADC tumors (87.4%) (FIG. 7A). Then, a correlation of OIP5 expression (positive versus negative) with various clinicopathological parameters was examined and found its significant correlation with histology (higher in non-ADCs; P<0.0001 by Fisher's exact test) and with tumor size (higher in pT2-T3; P=0.0318 by Fisher's exact test) and with smoking (higher in smoker; P=0.0187 by Fisher's exact test) (Table 3A). The Kaplan-Meier method indicated significant association between OIP5 positivity and shorter tumor-specific survival periods of NSCLC patients (P=0.0053 by the log-rank test; FIG. 7B). By univariate analysis, non-adenocarcinoma histology, larger tumor size (pT2-3), presence of lymph node metastasis (pN1-2), elderly (>65 years), male gender (female versus male), and OIP5 positivity were significantly related to poor tumor-specific survival of NSCLC patients (Table 3B). Furthermore, multivariate analysis using the Cox proportional hazard model indicated that pT stage, pN stage, age, and positive OIP5 staining were independent prognostic factors for NSCLC patients (Table 3B).

TABLE 3A
Association between OIP5-positivity in NSCLC tissues and patients'
characteristics (n = 336)

| | | OIP5 | OIP5 | |
|---|---|---|---|---|
| | Total | positive | negative | P-value: |
| | n = 336 | n = 249 | n = 87 | positive vs negative |
| Age (years) | | | | |
| <65 | 178 | 130 | 48 | 0.7084 |
| >=65 | 158 | 119 | 39 | |
| Gender | | | | |
| Female | 103 | 70 | 33 | 0.1049 |
| Male | 233 | 179 | 54 | |
| Histological type | | | | |
| ADC | 201 | 131 | 70 | <0.0001* |
| non-ADC | 135 | 118 | 17 | |
| pT factor | | | | |
| T1 | 140 | 95 | 45 | 0.0318* |
| T2 + T3 | 196 | 154 | 42 | |
| pN factor | | | | |
| N0 | 218 | 155 | 63 | 0.0917 |
| N1 + N2 | 118 | 94 | 24 | |
| smoking | | | | |
| non-smoker | 94 | 61 | 33 | 0.0187* |
| smoker | 242 | 188 | 54 | |

ADC, adenocarcinoma
non-ADC, squamous-cell carcinoma plus large-cell carcinoma and adenosquamous-cell carcinoma
*P < 0.05 (Fisher's exact test)

TABLE 3B
Cox's proportional hazards model analysis of prognostic factors in
patients with NSCLCs

| | Hazards | | Unfavorable/ | |
|---|---|---|---|---|
| Variables | ratio | 95% CI | Favorable | P-value |
| Univariate | | | | |
| analysis | | | | |
| OIP5 | 1.854 | 1.193-2.882 | Positive/Negative | 0.0061* |
| Age (years) | 1.557 | 1.103-2.199 | >=65/65> | 0.0119* |
| Gender | 1.619 | 1.094-2.396 | Male/Female | 0.0159* |
| Histological type | 1.428 | 1.021-1.998 | non-ADC/ADC | 0.0374* |
| pT factor | 2.4 | 1.610-3.513 | T2 + T3/T1 | <0.0001* |
| pN factor | 2.189 | 1.565-3.063 | N1 + N2/N0 | <0.0001* |
| smoking | 1.252 | 0.849-1.844 | smoker/ | 0.2567 |
| | | | non-smoker | |
| Multivariate | | | | |
| analysis | | | | |
| OIP5 | 1.812 | 1.144-2.869 | Positive/Negative | 0.0112* |
| Age (years) | 1.752 | 1.228-2.499 | >=65/65> | 0.002* |
| Gender | 1.357 | 0.878-2.095 | Male/Female | 0.1691 |
| Histological type | 0.863 | 0.586-1.271 | non-ADC/ADC | 0.4554 |
| pT factor | 1.87 | 1.250-2.796 | T2 + T3/T1 | 0.0023* |
| pN factor | 2.115 | 1.491-3.001 | N1 + N2/N0 | <0.0001* |

ADC, adenocarcinoma
non-ADC, squamous-cell carcinoma plus large-cell carcinoma and adenosquamous-cell carcinoma
*P < 0.05

On the other hand, positive staining of OIP5 was observed in 172 of 297 (57.9%) surgically resected esophageal cancers, whereas no staining was observed in any of the adjacent normal esophageal tissues (FIG. 7C). A correlation of OIP5 expression (positive versus negative) with various clinicopathological parameters was examined and found its significant correlation with tumor size (higher in pT2-T3; P=0.004 by Fisher's exact test) and with lymph node metastasis (higher in pN1-N2; P=0.0052 by Fisher's exact test) (Table 4A). The Kaplan-Meier analysis indicated significant association between OIP5 positivity and shorter tumor-specific survival periods of ESCC patients (P=0.0129 by the log-rank test; FIG. 7D). By univariate analysis, larger tumor size (pT2-3), lymph node metastasis positive (pN1-2), male gender, and OIP5 positivity were significantly related to poor tumor-specific survival of ESCC patients (Table 4B). In multivariate analysis, OIP5 status did not reach the statistically significant level as independent prognostic factor for surgically treated ESCC patients enrolled in this study (P=0.1015), while pT and pN stages as well as age did so, suggesting the relevance of OIP5 expression to these clinicopathological factors in esophageal cancer (Table 4B).

TABLE 4A
Association between OIP5 positivity in esophageal cancer tissues and
patients' characteristics (n = 297)

| | | OIP5 | OIP5 | |
|---|---|---|---|---|
| | Total | positive | negative | P-value: |
| | n = 297 | n = 172 | n = 125 | positive vs negative |
| Age (years) | | | | |
| <65 | 182 | 100 | 82 | 0.2277 |
| >=65 | 115 | 72 | 43 | |
| Gender | | | | |
| Female | 34 | 21 | 13 | 0.7136 |
| Male | 263 | 151 | 112 | |
| pT factor | | | | |
| T1 | 98 | 45 | 53 | 0.004* |
| T2 + T3 | 199 | 127 | 72 | |
| pN factor | | | | |
| N0 | 112 | 53 | 59 | 0.0052* |
| N1 + N2 | 185 | 119 | 66 | |

*P < 0.05 (Fisher's exact test)

TABLE 4B
Cox's proportional hazards model analysis of prognostic factors in
patients with esophageal cancers

| | Hazards | | Unfavorable/ | |
|---|---|---|---|---|
| Variables | ratio | 95% CI | Favorable | P-value |
| Univariate analysis | | | | |
| OIP5 | 1.516 | 1.090-2.111 | Positive/ | 0.0135* |
| | | | Negative | |
| Age (years) | 1.002 | 0.725-1.385 | >=65/65> | 0.9891 |
| Gender | 3.332 | 1.634-6.792 | Male/Female | 0.0009* |
| pT factor | 2.841 | 1.886-4.281 | T2 + T3/T1 | <0.0001* |
| pN factor | 4.036 | 2.676-6.086 | N1 + N2/N0 | <0.0001* |
| Multivariate analysis | | | | |
| OIP5 | 1.32 | 0.947-1.841 | Positive/ | 0.1015 |
| | | | Negative | |
| Gender | 2.903 | 1.423-5.926 | Male/Female | 0.0034* |
| pT factor | 1.953 | 1.281-2.977 | T2 + T3/T1 | 0.0019* |
| pN factor | 3.149 | 2.064-4.804 | N1 + N2/N0 | <0.0001* |

*P < 0.05

Effect of OIP5 on Cell Growth and Invation.

To assess whether upregulation of OIP5 plays a role in growth or survival of lung-cancer cells, siRNA against OIP5 (si-1 and -2) were transfected, along with two different control (siRNAs for LUC and, CNT) into LC319 and SBC-5 cells to suppress expression of endogenous OIP5 (FIG. 3A). The level of OIP5 expression in the cells transfected with si-1, si-2 was significantly reduced, in comparison with two control siRNAs (FIG. 3A, top panels). Cell viability and colony numbers measured by MTT and colony-formation assays were reduced significantly in the cells transfected with si-1 or si-2 in comparison with those transfected with control siRNA (FIG. 3A, middle panels).

To further examine a potential role of OIP5 in tumorigenesis, plasmids designed to express OIP5 (pcAGGSn3FC-OIP5-Flag) were prepared and transfected into COS-7 cells. After confirmation of OIP5 expression by western-blot analysis (FIG. 3B, left top panels), MTT and colony-formation assays were carried out, and it was found that growth of the OIP5-COS-7 cells was promoted at a significant degree in comparison to the COS-7 cells transfected with the mock vector (FIG. 3B, left bottom and right panels). There was also a remarkable tendency in the COS-7-OIP5 cells to form larger colonies than the control cells (FIG. 3B, left bottom panels), implying that OIP5 has an oncogenic activity in mammalian cells.

Matrigel invasion assays was done to determine whether OIP5 might play some role in cellular invasive ability. Invasion of COS-7-OIP5 cells through Matrigel was significantly enhanced, compared with the control cells transfected with mock plasmids (FIG. 9B). These results independently suggest that OIP5 could contribute to the highly malignant phenotype of cancer cells.

Stabilization of OIP5 Protein Through its Interaction with Raf1.

To elucidate the biological importance of OIP5 activation in carcinogenesis, proteins that would interact with OIP5 in cancer cells were attempted to be identified. A previous report about an exhaustive yeast two-hybrid screening using N-terminal regulatory domain of human Raf1 as “bait” indicated that OIP5 was one of 20 candidate interaction partners of Raf1 (Yuryev A and Wennogle L P. Genomics 2003; 81:112-25.), although their physiological interaction and function in mammalian cells were not clarified yet. Raf1 is well known to be activated in a wide range of tumor types, and this triggers a cascade of responses, from cell growth and proliferation to survival and motility (Yuryev A and Wennogle L P. Genomics 2003; 81:112-25.). Therefore, firstly whether OIP5 could be physiologically associated with Raf1 was examined. Immunoprecipitation of OIP5 in COS-7 cells transfected with Flag-tagged OIP5 expressing plasmids using anti-Flag antibody followed by immunoblotting with anti-Raf1 antibodies indicated the interaction of exogenous OIP5 with endogenous Raf1 (FIG. 8A). Next, the direct interaction of OIP5 with Raf1 protein was confirmed by pull-down assay. Pull-down of OIP5 protein was carried out using anti-His antibody and incubated mixture of His-tagged OIP5 and GST-fused recombinant Raf1 proteins. OIP5-binding Raf1 protein was detected by subsequent western blotting using polyclonal antibody to Raf1 (FIG. 9C). Next, OIP5 expression in human lung-cancer cell lines were examined by semi-quantitative RT-PCR experiments (FIG. 9D) and western-blotting (FIG. 8B), and found co-expression of OIP5 and Raf1 in most of lung-cancer cells examined, suggesting the possibility of a complex formation of these two proteins in lung cancer cells.

To further assess whether expression of Raf1 plays a role in the regulation of OIP5 function in cancer cells, the biological significance of the Raf1 was examined using siRNAs against Raf1. To further assess the effect of Raf1 on OIP5 protein function in cancer cells, the level of endogenous OIP5 protein after transfection of siRNA for Raf1 to SBC-5 cells was measured. Interestingly, the level of OIP5 protein was decreased in cells treated with si-Raf1, while the expression level of OIP5 mRNA was not changed (FIG. 8C, left panels). On the contrary, overexpression of Raf1 resulted in the increase of expression level of OIP5 protein, while the expression level of OIP5 was not varied (FIG. 8C, right panels), indicating a possibility that OIP5 protein stability is regulated by the Raf1.

2. Discussion

Despite improvement of modern surgical techniques and adjuvant chemoradiotherapy, prognosis of lung cancer and ESCC is known to be poor among malignant tumors. Several molecular-targeting drugs have been developed and proved their efficacy in cancer therapy; however, the proportion of patients showing good response is still limited (Ranson M, et al., J Clin Oncol 2002; 20: 2240-50). Accordingly, there is an urgent need to develop new anti-cancer agents that will be highly specific to malignant cells, with minimal or no adverse reactions. A genome-wide expression profile analysis of 101 lung cancers and 19 ESCC cells after enrichment of cancer cells was performed by laser microdissection, using a cDNA microarray containing 27,648 genes (Daigo Y and Nakamura Y, Gen Thorac Cardiovasc Surg 2008; 56:43-53, Kikuchi T, et al., Oncogene 2003; 22:2192-205, Kakiuchi S, et al., Mol Cancer Res 2003; 1:485-99, Kakiuchi S, et al., Hum Mol Genet 2004; 13:3029-43, Kikuchi T, et al., Int J Oncol 2006; 28:799-805, Taniwaki M, et al., Int J Oncol 2006; 29:567-75, and Yamabuki T, et al., Int J Oncol 2006; 28:1375-84). Present inventors have undertaken a strategy that combines screening of candidate molecules by genome-wide expression analysis with high-throughput screening of loss-of-function effects, using the RNAi technique, and have taken the systematic analysis of protein expression among hundreds of clinical samples on tissue microarrays (Suzuki C, et al., Cancer Res 2003; 63:7038-41, Ishikawa N, et al., Clin Cancer Res 2004; 10:8363-70, Kato T, et al., Cancer Res 2005; 65:5638-46, Furukawa C, et al., Cancer Res 2005; 65:7102-10, Ishikawa N, et al., Cancer Res 2005; 65:9176-84, Suzuki C, et al., Cancer Res 2005; 65:11314-25, Ishikawa N, et al., Cancer Sci 2006; 97:737-45, Takahashi K, et al. Cancer Res 2006; 66:9408-19, Hayama S, et al., Cancer Res 2006; 66:10339-48, Kato T, et al., Clin Cancer Res 2007; 13:434-42, Suzuki C, et al., Mol Cancer Ther 2007; 6:542-51, Yamabuki T, et al., Cancer Res 2007; 67:2517-25, Hayama S, et al., Cancer Res 2007; 67:4113-22, Kato T, et al., Cancer Res 2007; 67:8544-53, Taniwaki M, et al., Clin Cancer Res 2007; 13:6624-31, Ishikawa N, et al., Cancer Res 2007; 67:11601-11, Mano Y, et al., Cancer Sci 2007; 98:1902-13, Suda T, et al., Cancer Sci 2007; 98:1803-8, Kato T, et al., Clin Cancer Res 2008; 14:2363-70, and Mizukami Y, et al., Cancer Sci 2008; 99:1448-54). The results have shown that OIP5 is frequently over-expressed in clinical lung and esophageal cancers samples, and cell lines, and that the gene product is indispensable for survival/growth and promotion of the malignant potential of cancer cells.

OIP5 protein encodes a 229-amino-acid protein with a coiled-coil domain. OIP5 was found by yeast two-hybrid analysis to interact with Neisseria Gonorrhoeae opacity-associated (Opa) proteins (Williams, J. M., et al., Mol Microbiol 1998; 27(1): 171-86), suggesting its involvement in gonococcal adhesion and invasion to human epithelial cells. OIP5 was also found to be interacted with Raf1 by an exhaustive yeast two-hybrid analysis (Yuryev, A. and L. P. Wennogle, Genomics 2003; 81(2): 112-25). OIP5 is suggested to be involved in cell cycle exit as a nuclear protein through its binding to Lamina-associated polypeptide (LAP2a) (Naetar, N., et al., J Cell Sci 2007; 120(Pt 5): 737-47).

In the present study, it was demonstrated that OIP5 gene is frequently over-expressed in lung and esophageal cancers, and may play an important role in the development of those cancers.

Knockdown of OIP5 expression by siRNA in lung cancer cells resulted in suppression of cell growth. Moreover, clinicopathological evidence obtained through our tissue-microarray experiments indicated that NSCLC patients with OIP5-positive tumors had shorter cancer-specific survival periods than those with OIP5-negative tumors. Importantly, Raf1 could interact with and stabilize OTP5 in cancer cells. The results obtained by in vitro and in vivo assays strongly suggested that OIP5 is likely to be an important growth factor and be associated with a more malignant phenotype of lung-cancer cells. Further investigations of OIP5 pathway may lead to a better understanding of the mechanisms of oncogenes activation in carcinogenesis. Additionally, since these results indicate that OIP5 plays a significant role in cancer cell growth/survival as one of the components of the Raf1 pathway, selective targeting of functional interaction between Raf1 and OIP5 could be a promising therapeutic strategy, although further investigations of OIP5 pathway that could lead to a better understanding of the mechanisms of OIP5 oncogene activation is necessary.

Because OIP5 should be classified as one of the typical cancer testis antigens, selective inhibition of OIP5 activity by molecular targeted agents could be a promising therapeutic strategy that is expected to have a powerful biological activity against cancer with a minimal risk of adverse events.

In summary, OIP5 may play an important role in the growth of lung and esophageal cancers by interacting with Raf1. OIP5 over-expression in resected specimens may be a useful index for application of adjuvant therapy to the patients who are likely to have poor prognosis. In addition, the data strongly suggests the possibility of designing new anti-cancer drugs and cancer vaccines to specifically target the OIP5 for human cancer treatment.

### INDUSTRIAL APPLICABILITY

The gene-expression analysis of cancers described herein using the combination of laser-capture dissection and genome-wide cDNA microarray has identified specific genes as targets for cancer prevention and therapy. Based on the expression of a differentially expressed gene, OIP5, the present invention provides a molecular diagnostic marker for identifying and detecting cancer, in particular, lung and/or esophageal cancer.

The data provided herein add to a comprehensive understanding of cancers, facilitate development of novel diagnostic strategies, and provide clues for identification of molecular targets for therapeutic drugs and preventative agents. Such information contributes to a more profound understanding of tumorigenesis, and provide indicators for developing novel strategies for diagnosis, treatment, and ultimately prevention of cancers.

All patents, patent applications, and publications cited herein are incorporated by reference in their entirety.

Furthermore, while the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method for diagnosing lung and/or esophageal cancer, said method comprising the steps of:
(a) determining the expression level of an OIP5 gene in a subject-derived biological sample by any one of the method select from the group consisting of:
(i) detecting mRNA of the OIP5 gene,
(ii) detecting a protein encoded by the OIP5 gene, and
(iii) detecting a biological activity of a protein encoded by the OIP5 gene; and
(b) correlating an increase in the expression level determined in step (a) as compared to a normal control level of the gene to the presence of lung and/or esophageal cancer.

2. The method of claim 1, wherein the expression level determined in step (a) is at least 10% greater than the normal control level.

3. The method of claim 1, wherein the expression level determined in step (a) is determined by detecting the binding of an antibody against the OIP5 protein.

4. The method of claim 1, wherein the subject-derived biological sample comprises biopsy.

5. A method for assessing or determining the prognosis of a patient with lung and/or esophageal cancer, which method comprises the steps of:
(a) detecting the expression level of an OIP5 gene in a patient-derived biological sample;
(b) comparing the detected expression level to a control level; and
(c) determining the prognosis of the patient based on the comparison of (b).

6. The method of claim 5, wherein the control level is a good prognosis control level and an increase of the expression level compared to the control level is determined as poor prognosis.

7. The method of claim 6, wherein the increase is at least 10% greater than the control level.

8. The method of claim 5, wherein the expression level is determined by any one method selected from the group consisting of:
(a) detecting mRNA of the OIP5 gene;
(b) detecting a protein encoded by the OIP5 gene; and
(c) detecting a biological activity of a protein encoded by the OIP5 gene.

9. The method of claim 5, wherein the patient-derived biological sample comprises biopsy.

10. A kit for diagnosing lung and/or esophageal cancer or assessing or determining the prognosis of a patient suffering from lung and/or esophageal cancer, which comprises a reagent selected from the group consisting of:
(a) a reagent for detecting mRNA of an OIP5 gene;
(b) a reagent for detecting a protein encoded by an OIP5 gene; and
(c) a reagent for detecting a biological activity of a protein encoded by an OIP5 gene.

11. The kit of claim 10, wherein the reagent is a probe to a gene transcript of the gene.

12. The kit of claim 10, wherein the reagent is an antibody against the protein encoded by the gene.

13. An isolated double-stranded molecule that, when introduced into a cell, inhibits in vivo expression of an OIP5 gene as well as cell proliferation, said molecule comprising a sense strand and an antisense strand complementary thereto, said strands hybridized to each other to form the double-stranded molecule, wherein the sense strand comprises a nucleotide sequence corresponding to a contiguous sequence from SEQ ID NO: 13.

14. The double-stranded molecule of claim 13, wherein the sense strand comprises the sequence corresponding to a target sequence selected from the group consisting of SEQ ID NOs: 11 and 12.

15. The double-stranded molecule of claim 14, wherein the double stranded molecule is an oligonucleotide of between about 19 and about 25 nucleotides in length.

16. The double-stranded molecule of claim 13, which consists of a single polynucleotide comprising both the sense and antisense strands linked by an intervening single-strand.

17. The double-stranded molecule of claim 16, which has the general formula 5′-[A]-[B]-[A′]-3′, wherein [A] is the sense strand comprising a sequence corresponding to a target sequence selected from the group consisting of SEQ ID NOs: 11 and 12, [B] is the intervening single-strand consisting of 3 to 23 nucleotides, and [A′] is the antisense strand comprising a complementary sequence to [A].

18. A vector encoding the double-stranded molecule of claim 13 to 17.

19. A method for treating or preventing a cancer expressing an OIP5 gene, wherein the method comprises the step of administering at least one isolated double-stranded molecule of claims 13 to 17 or vector of claim 18.

20. The method of claim 19, wherein the cancer to be treated is lung and/or esophageal cancer.

21. A composition for treating or preventing a cancer expressing an OIP5 gene, wherein composition comprised at least one isolated double-stranded molecule of claims 13 to 17 or vector of claim 18.

22. The composition of claim 21, wherein the cancer to be treated is lung and/or esophageal cancer.

23. A method of screening for a candidate compound for treating or preventing a cancer associated with the over-expression of an OIP5 gene, or inhibiting said cancer cells growth, said method comprising the steps of:
(a) contacting a test compound with a polypeptide encoded by a polynucleotide of an OIP5 gene;
(b) detecting the binding activity between the polypeptide and the test compound; and
(c) selecting the test compound that binds to the polypeptide.

24. A method of screening for a candidate compound for treating or preventing a cancer associated with the over-expression of an OIP5 gene, or inhibiting said cancer cells growth, said method comprising the steps of:
(a) contacting a test compound with a polypeptide encoded by a polynucleotide of an OIP5 gene;
(b) detecting a biological activity of the polypeptide of step (a); and
(c) selecting the test compound that suppresses the biological activity of the polypeptide encoded by the polynucleotide of the OIP5 gene as compared to the biological activity of said polypeptide detected in the absence of the test compound.

25. The method of claim 24, wherein the biological activity is the facilitation of the cell proliferation.

26. A method of screening for a candidate compound for treating or preventing s cancer associated with the over-expression of an OIP5 gene, or inhibiting said cancer cells growth, said method comprising the steps of:
(a) contacting a test compound with a cell expressing an OIP5 gene and
(b) selecting the test compound that reduces the expression level of the OIP5 gene in comparison with the expression level detected in the absence of the test compound.

27. A method of screening for a candidate compound for treating or preventing a cancer associated with the over-expression of an OIP5 gene, or inhibiting said cancer cells growth, said method comprising the steps of:
(a) contacting a test compound with a cell into which a vector, comprising the transcriptional regulatory region of the OIP5 gene and a reporter gene that is expressed under the control of the transcriptional regulatory region, has been introduced;
(b) measuring the expression or activity of said reporter gene; and
(c) selecting the test compound that reduces the expression or activity level of said reporter gene as compared to a control.

28. A method of screening for a candidate compound for treating or preventing a cancer associated with the over-expression of an OIP5 gene, or inhibiting said cancer cells growth, said method comprising steps of:
(a) contacting an OIP5 polypeptide or a functional equivalent thereof with a Raf1 polypeptide or a functional equivalent thereof in the presence of a test compound;
(b) detecting a binding level between the polypeptides;
(c) comparing the binding level detected in the step (b) with those detected in absence of the test compound; and
(d) selecting the test compound that reduces the binding level comparing with those detected in absence of the test compound in step (c).

29. The method of claim 28, wherein the functional equivalent of OIP5 comprises Raf1-binding domain.

30. A method of screening for a candidate compound for treating or preventing a cancer associated with the over-expression of an OIP gene, or inhibiting said cancer cells growth, said method comprising steps of:
(a) contacting an OIP5 polypeptide or a functional equivalent thereof with a Raf1 polypeptide or a functional equivalent thereof in the presence of a test compound under a suitable condition for phosphorylation;
(b) detecting the phosphorylation level of the OIP5 polypeptide; and
(c) selecting the test compound that reduces the phosphorylation level of the OIP5 polypeptide as compared to the phosphorylation label detected in the absence of the test compound.

31. A method of any one of claims 23, 24, 26, 27, 28 or 29, wherein the cancer is selected from lung cancer and esophageal cancer.


SEQUENCE LISTING (18 sequences)

SEQ ID NO: 1 - DNA, 20 bp, Artificial
  An artificially synthesized primer for PCR
cttcaagaatggaggggaaa

SEQ ID NO: 2 - DNA, 24 bp, Artificial
  An artificially synthesized primer for PCR
gtattcataacaactgctccatgc

SEQ ID NO: 3 - DNA, 21 bp, Artificial
  An artificially synthesized primer for PCR
gaggtgatagcattgctttcg

SEQ ID NO: 4 - DNA, 21 bp, Artificial
  An artificially synthesized primer for PCR
caagtcagtgtacaggtaagc

SEQ ID NO: 5 - DNA, 20 bp, Artificial
  An artificially synthesized primer for Northern blot
ccagtgacaaaatggtgtgc

SEQ ID NO: 6 - DNA, 24 bp, Artificial
  An artificially synthesized primer for Northern blot
gtattcataacaactgctccatgc

SEQ ID NO: 7 - DNA, 19 bp, Artificial
  An artificially synthesized for siRNA
cguacgcggaauacuucga

SEQ ID NO: 8 - DNA, 19 bp, Artificial
  An artificially synthesized for siRNA
ugguuuacaugucgacuaa

SEQ ID NO: 9 - DNA, 21 bp, Artificial
  An artificially synthesized for siRNA
cggcaucgcucacguuguguu

SEQ ID NO: 10 - DNA, 21 bp, Artificial
  An artificially synthesized for siRNA
gugacaaaauggugugcuauu

SEQ ID NO: 11 - DNA, 19 bp, Artificial
  An artificially synthesized target sequence for siRNA
cggcatcgctcacgttgtg

SEQ ID NO: 12 - DNA, 19 bp, Artificial
  An artificially synthesized target sequence for siRNA
gtgacaaaatggtgtgcta

SEQ ID NO: 13 - DNA, 1249 bp, Homo sapiens
gagctgtgcttggggcggggcctggcgtgtattcgaaaggaaggcgccggctgcgggaagatggcggctcagccgctgcggcatcgctcacgttgtgcaacgccgccccggggggacttttgtggtggcactgagagggcgattgaccaagcttcttttacgacctccatggagtgggatacgcaggtggtgaaggggtcctcgccgctcggccccgcagggctgggggctgaggagccagccgccggcccgcagctgccgtcttggctgcagcctgagaggtgcgctgtgttccagtgcgcacagtgtcacgcagtgctcgccgactcggtgcacctcgcctgggacctgtcgcggtccctcggggccgtggtcttctccagagttacaaataacgtcgttttggaagcgcccttcctagttggcattgaaggttcactcaaaggcagtacttacaaccttttattctgtggttcttgtgggattcccgttggtttccatctgtattctacccatgctgccctggctgccttgagaggtcacttctgcctttccagtgacaaaatggtgtgctatctcttaaaaacaaaagccatagtaaatgcatcagagatggatattcaaaatgttcctctatcagaaaagattgcagagctgaaagagaagatagtgctaacgcacaatcgcttaaaatcactaatgaagattctgagtgaagtgactcctgaccagtccaagccagaaaactgatcctgtaccaaagcttgagtgtcaggttcaggctttattgctgtcttcaacaacaggtgctgcttagtcatttcttgaaaaagattggcttcaagaatggaggggaaatgcagtttctatttacctttaggctgattttccaaattatttgtgaagctgtttttagaagatgagagactaaggattcttctcttttatagctatttgccttaagaacttactttagattcttattgaattcataatacttatctctgaaaatgtctttgactgtaaatttaggaattaagatgcagagtcccatgtgtcctctgatctaaagttgcatggttggtctgaaaatagagttgggcttaatgttgacttctattactcctgcatggagcagttgttatgaatactaatacatcactttttaacttctgtaaaatacagatcataatattctataggtaatgtttaataaattgcctgaataatatacaaaaaaaaaaaaaaa

SEQ ID NO: 14 - protein, 229 aa, Homo sapiens
MAAQPLRHRSRCATPPRGDFCGGTERAIDQASFTTSMEWDTQVVKGSSPLGPAGLGAEEPAAGPQLPSWLQPERCAVFQCAQCHAVLADSVHLAWDLSRSLGAVVFSRVTNNVVLEAPFLVGIEGSLKGSTYNLLFCGSCGIPVGFHLYSTHAALAALRGHFCLSSDKMVCYLLKTKAIVNASEMDIQNVPLSEKIAELKEKIVLTHNRLKSLMKILSEVTPDQSKPEN

SEQ ID NO: 15 - DNA, 19 bp, Artificial
  An artificially synthesized for siRNA
gcaaagaacaucauccaua

SEQ ID NO: 16 - DNA, 19 bp, Artificial
  An artificially synthesized for siRNA
gacaugaaauccaacaaua

SEQ ID NO: 17 - DNA, 3291 bp, Homo sapiens
  CDS (416)..(2362)
agaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaaaaaaaaaaaaaaaaaa

SEQ ID NO: 18 - protein, 648 aa, Homo sapiens [sequence omitted]